(19) United States
(12) Patent Application Publication
(10) Pub. No.: US 2026/0062400 A1
(43) Pub. Date: Mar. 5, 2026

(54) SYSTEMS AND METHODS FOR THE SYNTHESIS OF HYDROXAMIC ACIDS

(71) Applicant: Kennesaw State University Research And Service Foundation, Inc., Kennesaw, GA (US)

(72) Inventors: Carl Jacky Saint-Louis, Marietta, GA (US); Albert D. Campbell, Kennesaw, GA (US); Ophelia Adjei-sah, Kennesaw, GA (US)

(21) Appl. No.: 19/314,922

(22) Filed: Aug. 29, 2025

Related U.S. Application Data
(60) Provisional application No. 63/688,682, filed on Aug. 29, 2024.

(51) Int. Cl. C07D 333/22; C07C 259/04; C07D 409/14

(52) U.S. Cl. CPC C07D 333/22; C07C 259/04; C07D 409/14

(57) ABSTRACT

The present disclosure provides a photolabile protecting group compound comprising a thiophene ring, a nitrobenzyl group having a nitro group in an ortho position relative to a benzylic carbon, an alkyne linker connecting the thiophene ring to the nitrobenzyl group, and a hydroxamic acid moiety attached to the benzylic carbon through an oxygen atom, wherein upon irradiation with ultraviolet light, the compound undergoes photolysis to release the hydroxamic acid and generate a fluorescent nitrosoketone byproduct. The thiophene ring is connected to the nitrobenzyl group through a 2-position of the thiophene ring. The nitrobenzyl group has a methyl group attached to the benzylic carbon. The compound has an absorption maximum between 340 nm and 360 nm, and upon photolysis, the fluorescent nitrosoketone byproduct has an emission maximum between 440 nm and 500 nm with a 3-fold to 4-fold increase in fluorescence intensity that is observable with a naked eye.

### CROSS-REFERENCE TO RELATED APPLICATIONS

This application is a Nonprovisional of and claims the benefit of provisional Ser. No. 63/688,682, filed Aug. 29, 2024, which is hereby incorporated by reference in its entirety for any and all purposes.

### GOVERNMENT RIGHTS

This invention was made with government support under (NSF-LEAPS-MPS) (CHE2137454) awarded by the National Science Foundation-Launching Early-Career Academic Pathways in the Mathematical and Physical Sciences. The government has certain rights in the invention.

### BACKGROUND

Photolabile protecting groups (PPGs) have become valuable tools in organic synthesis and chemical biology, offering a mild and selective method for removing protective groups from sensitive molecules. Unlike traditional deprotection methods that rely on harsh acidic, basic, reductive, or oxidative conditions, PPGs enable the controlled release of protected functional groups using light as the activating stimulus. This light-triggered approach provides spatial and temporal control over deprotection reactions while minimizing potential degradation of the target molecule.

The ortho-nitrobenzyl (o-NB) group represents one of the most widely studied classes of PPGs since its introduction in the 1960s. These compounds undergo photolysis through a well-characterized mechanism involving intramolecular hydrogen atom transfer, formation of aci-nitro intermediates, and subsequent cyclization and fragmentation steps.

Despite their utility, conventional PPGs present challenges in monitoring the progress of photolysis reactions. Real-time detection of product release during and after photolysis remains difficult with most existing PPG systems. The ability to directly monitor uncaging events through observable changes, such as fluorescence signals, would provide substantial advantages for tracking reaction progress and quantifying released products. Currently, only limited examples of PPGs exhibit pro-fluorescent properties that enable direct detection of uncaging events.

Hydroxamic acids represent another area where synthetic challenges persist. These compounds find applications as metal chelators, precursors for pharmaceutical agents, and components in various materials. However, the synthesis of hydroxamic acids often proves difficult due to their high reactivity and tendency to form complex mixtures under conventional reaction conditions. Traditional synthetic approaches frequently result in low yields and challenging purification processes, highlighting the need for alternative synthetic strategies that can provide cleaner reaction pathways and improved product isolation.

Improvements are needed.

### SUMMARY

The development of PPGs that combine effective photolysis with real-time monitoring capabilities could address multiple synthetic challenges simultaneously. Such systems would enable both the controlled synthesis of difficult-to-prepare compounds and provide immediate feedback on reaction progress through observable changes in optical properties.

Described herein are systems and methods for synthesis, characterization, and application of visible light-absorbing, fluorescent-quenching thiophene-based o-nitrobenzyl photolabile protecting groups for the synthesis of hydroxamic acids. As used herein, visible light may include electromagnetic radiation (light) having 380 nm-750 nm wavelength.

Described herein are systems and methods for development of a photolabile visible light absorbing, fluorescent-quenching photolabile protecting group.

Described herein are systems and methods for development and design of a visible light absorbing, fluorescent-quenching photolabile protecting group for the synthesis of hydroxamic acids. Described herein are systems and methods for development and design of visible light absorbing, fluorescent-quenching photolabile protecting group for light regulation of biomolecules and cleavage of reactive functional groups. Described herein are systems and methods for development of efficient visible light absorbing photolabile drug protecting groups.

The systems and methods described herein may be used with additional reactive functional groups that are difficult to synthesize.

There is a great need for photolabile protective groups (PPGs) that can produce an easy means to quantify difficult-to-synthesize compounds with reactive functional groups such hydroxamic acids (HAs) due to difficulty with purification. HAs are an essential class of organic compounds that can be used as precursors in the development of chemotherapeutic drugs for various cancers and as potent metal chelators in the removal of harmful metals from seawater. However, there is a significant limitation with the synthesis and purification of HAs due to their high reactivity, resulting in many polysubstituted by-products. This problem is addressed herein. The systems and methods described herein include design, development, and synthesization of the first example of a series of visible light-absorbing, fluorescent-quenching thiophene-based o-nitrobenzyl photoremovable groups. Photolysis of the visible light-absorbing PPGs leads to the formation of a di-nitrosoketone byproduct which quenches the fluorescence and can be used to quantify the amount of HAs cleaved in real-time by measuring the loss of emission and absorption detectable with the naked eye, allowing determination of when HA cleavage/synthesis is complete. This new type of PPG will also aid in the development of future visible light-absorbing PPGs that are utilized to cleave other difficult-to-synthesize functional groups and may be used in drug delivery systems.

A well-known solution to the obstacle above is the use of protecting groups (PGs) to temporarily block the reactivity of highly reactive functional groups such as HAs.

Nevertheless, PGs must be removed under harsh conditions such as acidic, basic, or oxidative, which can degrade the protected product. Selective PG removal is also problematic when multiple PGs under similar conditions are applied to the same compound. Modern drug delivery system includes a pill that you swallow or a vaccine that is injected. Herein, only visible light is used to release or synthesize the compound with reactive functional groups.

PGs must be removed under harsh conditions such as acidic, basic, or oxidative, which can degrade the protected product. The systems and methods described herein provide a valuable tool to remedy this problem and the use of harsh conditions because only visible light is required for selectively cleaved the reactive functional groups.

The systems and methods described herein include design, development, and synthesization of the first example of a series of visible light-absorbing, fluorescent-quenching thiophene-based o-nitrobenzyl photoremovable groups capable of producing a diagnostic di-nitrosoketone byproduct which quenches the fluorescence detectable with the naked eye, allowing a determination of when HA cleavage/synthesis is complete, allowing a determination of when the functional group is cleavage/synthesis.

Described herein are systems and methods for synthesis, characterization, and application of new pro-flourescent thiophene-based o-nitrobenzyl photolabile protecting groups for the synthesis of hydroxamic acids.

Described herein are systems and methods for development of a photolabile drug delivery system. Described herein are systems and methods for development of an efficient visible light sensitive photolabile drug delivery system. Described herein are systems and methods for development of a photolabile protecting group for the synthesis of hydroxamic acids and synthesis of cancer drug (histone deacetylase inhibitors). Described herein are systems and methods for development of visible light sensitive photolabile protecting group for the synthesis of hydroxamic acids and synthesis of cancer drug (histone deacetylase inhibitors).

The systems and methods described herein may be used for testing in a biological system.

There is a great need for photolabile protective groups (PPGs) that can produce an easy means to quantify difficult-to-synthesize compounds such hydroxamic acids due to difficulty with purification. Hydroxamic acids (HAs) are an essential class of organic compounds that can be used as precursors in the development of chemotherapeutic drugs for various cancers and as potent metal chelators in the removal of harmful metals from seawater. However, there is a significant limitation with the synthesis and purification of HAs due to their high reactivity, resulting in many polysubstituted by-products. This problem is addressed herein. The systems and methods described herein include design, development, and synthesization of the first example of a pro-fluorescent thiophene-based o-nitrobenzyl photoremovable groups capable of producing a diagnostic nitroso-fluorescent byproduct detectable with the naked eye, allowing a determination of HA cleavage/synthesis is complete. This PPG can also be used to measure the amount of product released in real time as a result of the light-induced concentration jump. This new type of PPG will also aid in the development of future PPGs that are utilized to cleave other difficult-to-synthesize functional groups and may be used in drug delivery systems.

A well-known solution to the obstacle above is the use of protecting groups (PGs) to temporarily block the reactivity of highly reactive functional groups such as HAs. Nevertheless, PGs must be removed under harsh conditions such as acidic, basic, or oxidative, which can degrade the protected product. Selective PG removal is also problematic when multiple PGs under similar conditions are applied to the same compound. Modern drug delivery system includes a pill that you swallow or a vaccine that is injected. Herein, only light is used to release or synthesize the compound or to deliver the drug.

PGs must be removed under harsh conditions such as acidic, basic, or oxidative, which can degrade the protected product. The systems and methods described herein provide a valuable tool to remedy this problem and the use of harsh conditions because only light is required for selectively cleaved the functional groups or delivery the drug.

The systems and methods described herein include design, development, and synthesization of the first example of a pro-fluorescent thiophene-based o-nitrobenzyl photoremovable groups capable of producing a diagnostic nitroso-fluorescent byproduct detectable with the naked eye, allowing a determination of when HA cleavage/synthesis is complete. The photolabile protecting group described herein produces a diagnostic nitroso-fluorescent byproduct detectable with the naked eye, allowing a determination of when the functional group is cleavage/synthesis or when drug is delivered.

### BRIEF DESCRIPTION OF THE DRAWINGS

FIG. 1 shows two stages involved in synthesis of BNPETs 1 and 2.

FIG. 2A shows a graph illustrating absorbance spectral overlay of BNPET 1 in 9:1 MeCH/H₂0 with blowup of isosbestic points.

FIG. 2B shows a graph illustrating BNPET 1 showing uncleaved PPG and photo-release products after irradiation.

FIG. 3A shows a graph illustrating absorbance spectral overlay of BNPET 2 in 9:1 MeCH/H₂0 with blowup of isosbestic points.

FIG. 3B shows a graph illustrating BNPET 2 showing uncleaved PPG and photo-release products after irradiation.

FIG. 4A shows a graph illustrating emission spectral overlay showing decrease in emission intensity of BNPET 1 in 9:1 MeCH/H₂0.

FIG. 4B shows a side-by-side picture of uncleaved PPG (left) and photo-release products after photolysis (right).

FIG. 5A shows a graph illustrating emission spectral overlay showing decrease in emission intensity of BNPET 2 in 9:1 MeCH/H₂0.

FIG. 5B shows a side-by-side picture of uncleaved PPG (left) and photo-release products after photolysis (right).

FIG. 6 shows ¹H NMR spectral overlay of BNPET 1 stability test over thirty days.

FIG. 7 shows ¹H NMR spectral overlay of BNPET 1 stability test over thirty days.

FIG. 8 shows synthesis of novel NPET PPGs 1 and 2 with two carboxylic acid derivatives.

FIG. 9A shows a graph illustrating absorbance spectral overlay of NPET 1 (concentration=10⁴ M) in 9:1 MeCH:H₂0 with blowup of isosbestic points recorded at different times during phtotolysis upon irradiation at 365 millimeters(mm).

FIG. 9B shows a graph illustrating absorbance spectral overlay of NPET 1 with blowup of isosbestic points recorded after irradiation at 365 mm showing the newly formed nitrosoketone byproduct.

FIG. 10A shows a graph illustrating absorbance spectral overlay of NPET 2 (concentration=10⁴ M) in 9:1 MeCH:H₂0 with blowup of isosbestic points recorded at different times during phtotolysis upon irradiation at 365 mm.

FIG. 10B shows a graph illustrating absorbance spectral overlay of NPET 2 with blowup of isosbestic points recorded after irradiation at 365 mm showing the newly formed nitrosoketone byproduct.

FIG. 11A shows a graph showing NPET 1 (concentration=10⁴ M) in 9:1 MeCH:H₂0 recorded at different times during phtotolysis upon irradiation at 365 mm.

FIG. 11B shows a side-by-side picture of uncleaved NPET 1 (left) and the irradiated sample (right).

FIG. 12A shows a graph showing NPET 2 (concentration=10⁴ M) in 9:1 MeCH:H₂0 recorded at different times during phtotolysis upon irradiation at 365 mm.

FIG. 12B shows a side-by-side picture of uncleaved NPET 2 (left) and the irradiated sample (right).

FIG. 13 shows ¹H NMR spectral overlay (400 MHz) of NPET 1 stability test over thirty at ambient light.

FIG. 14 shows ¹H NMR spectral overlay (400 MHz) of NPET 2 stability test over thirty at ambient light.

FIG. 15 shows a set up for UV-Vis and emission photolysis for NPETs 1 and 2.

FIG. 16 shows a set up for ¹H-NMR photolysis for NPETs 1 and 2.

FIG. 17 shows a representation of 1-(2-nitro-5-(thiophen-2-ylethynyl)phenyl)ethan-1-one (4).

FIG. 18 shows a representation of 1-(2-nitro-5-(thiophen-2-ylethynyl)phenyl)ethan-1-ol (5).

FIG. 19 shows a representation of 2-(1-(2-nitro-5-(thiophen-2-ylethynyl)phenyl)ethoxy)isoindoline-1,3-dione (6).

FIG. 20 shows a representation of O-(1-(2-nitro-5-(thiophen-2-ylethynyl)phenyl)ethyl)hydroxylamine (7).

FIG. 21 shows a representation of 5-(((benzyloxy)carbonyl)amino)pentanoic acid (8).

FIG. 22 shows a representation of 4-(((tetrahydro-2H-pyran-2-yl)oxy)methyl)benzoic acid (9).

FIG. 23 shows a representation of benzyl (5-((1-(2-nitro-5-(thiophen-2-ylethynyl) phenyl) ethoxy) amino)-5-oxopentyl) carbamate (NPET 1).

FIG. 24 shows a representation of 4-(hydroxymethyl)-N-(1-(2-nitro-5-(thiophen-2 ylethynyl) phenyl) ethoxy) benzamide (NPET 2).

FIG. 25 shows characterization of ¹H NMR spectrum (400 MHz) of 4 in CDCl₃ at 25° C.

FIG. 26 shows characterization of ¹³C {¹H} NMR (100 MHz) of 4 in CDCl₃ at 25° C.

FIG. 27 shows characterization of ¹H NMR spectrum (400 MHz) of 5 in CDCl₃ at 25° C.

FIG. 28 shows characterization of ¹³C {¹H} NMR (100 MHz) of 5 in CDCl₃ at 25° C.

FIG. 29 shows characterization of ¹H NMR spectrum (400 MHz) of 6 in CDCl₃ at 25° C.

FIG. 30 shows characterization of ¹³C {¹H} NMR (100 MHz) of 6 in CDCl₃ at 25° C.

FIG. 31 shows characterization of ¹H NMR spectrum (400 MHz) of 7 in CDCl3 at 25° C.

FIG. 32 shows characterization of ¹³C {¹H} NMR (100 MHz) of 7 in CDCl₃ at 25° C.

FIG. 33 shows characterization of ¹H NMR spectrum (400 MHz) of 8 in DMSO-d₆ at 25° C.

FIG. 34 shows characterization of ¹³C {¹H} NMR (100 MHz) of 8 in DMSO-d₆ at 25° C.

FIG. 35 shows characterization of ¹H NMR spectrum (300 MHz) of 9 in CDCl₃ at 25° C.

FIG. 36 shows characterization of ¹³C {¹H} NMR (75 MHz) of 9 in CDCl₃ at 25° C.

FIG. 37 shows characterization of ¹H NMR spectrum (400 MHz) of NPET 1 in CD₃CN at 25° C.

FIG. 38 shows characterization of ¹³C {¹H} NMR (100 MHz) of NPET 1 in CD₃CN at 25° C.

FIG. 39 shows characterization of ¹³C {¹H} DEPT 135 NMR (100 MHz) of NPET 1 in CD₃CN at 25° C.

FIG. 40 shows characterization of ¹H-¹H COSY spectra of NPET 1 in CD₃CN (400 MHz).

FIG. 41 shows characterization of HSQC NMR spectra of NPET 1 in CD₃CN (400 MHz).

FIG. 42 shows characterization of HMBC NMR spectra of NPET 1 in CD₃CN (400 MHz).

FIG. 43 shows characterization of ¹H NMR spectrum (400 MHz) of NPET 2 in CD₃CN at 25° C.

FIG. 44 shows characterization of ¹³C {¹H} NMR (100 MHz) of NPET 2 in CD₃CN at 25° C.

FIG. 45 shows characterization of ¹³C {¹H} DEPT 135 NMR (100 MHz) of NPET 2 in CD₃CN at 25° C.

FIG. 46 shows characterization of ¹H-¹H COSY spectra of NPET 2 in CD₃CN (400 MHz).

FIG. 47 shows characterization of HSQC NMR spectra of NPET 2 in CD₃CN (400 MHz).

FIG. 48 shows characterization of HMBC NMR spectra of NPET 2 in CD₃CN (400 MHz).

FIG. 49 shows ¹H NMR overlay photolysis of NPET 1 in CD₃CN:1M HCl (4:1 v/v) before (t=0 min) and after irradiation (t=8 mins) with a 365 nm UV lamp (30-watt 4-core LED). FIG. 49(A) shows ¹H-NMR cleavage trace from 8.5 parts per million (ppm)-5.00 ppm and FIG. 49(B) shows ¹H-NMR cleavage trace from 3.00 ppm-0.5 ppm. NPET 1 concentration=4.2 millimolar (mM).

FIG. 50 shows ¹H NMR overlay photolysis of NPET 2 in CD₃CN:1M HCl (4:1) before (t=0 min) and after irradiation (t=8 mins) with a 365 nm UV lamp (30-watt 4-core LED). FIG. 50(A) shows ¹H-NMR cleavage trace from 8.5 ppm-5.00 ppm and FIG. 50(B) ¹H-NMR cleavage trace from 3.00 ppm-0.5 ppm. NPET 2 concentration=5.14 mM.

FIG. 51 shows ¹H NMR spectral overlay (400 MHz) of NPET 1 stability test over 30 days at ambient conditions.

FIG. 52 shows ¹H NMR spectral overlay (400 MHz) of NPET 2 stability test over 30 days at ambient conditions.

FIG. 53 shows normalized ultraviolet visible (UV-vis) spectrum of NPET 1 in different solvents of varying polarity (concentration=10⁻⁶ M).

FIG. 54 shows UV-vis spectrum of NPET 1 in different solvents of varying polarity (concentration=10⁻⁶ M).

FIG. 55 shows normalized UV-vis spectrum of NPET 2 in different solvents of varying polarity (concentration=10⁻⁶ M).

FIG. 56 shows UV-vis spectrum of NPET 2 in different solvents of varying polarity (concentration=10⁻⁶ M).

FIG. 57 is a table showing experimental UV-visible absorbance (λₘₐₓ) and molar absorption coefficient (ε) for NPET 1 and 2 in various solvents. (NPET concentration=10⁻⁶ M in solution).

FIG. 58 shows normalized emission spectrum of NPET 1 in different solvents of varying polarity (concentration=10⁻⁵ M).

FIG. 59 shows emission spectrum of NPET 1 in different solvents of varying polarity (concentration=10⁻⁵ M).

FIG. 60 shows normalized emission spectrum of NPET 2 in different solvents of varying polarity (concentration=10⁻⁵ M).

FIG. 61 shows emission spectrum of NPET 2 in different solvents of varying polarity (concentration=10⁻⁵ M).

FIG. 62 is a table showing experimental emission (λₑₘ), QYs (Φ_{F}), and Stokes shift (nm) and (cm⁻¹) data for NPET 1 and 2 in various solvents. (NPET concentration=10⁻⁵ M in solution).

FIG. 63 shows a graph showing photolysis emission (λₑₘ), QYs (Φ_{F}), and Stokes shift (nm) and (cm⁻¹) data for NPET 1 and 2 in various solvents. (NPET concentration=10⁻⁵ M in solution).

FIG. 64 shows a graph showing photolysis emission spectra of NPET 1.

FIG. 65 shows a graph showing photolysis absorbance of NPET 2.

FIG. 66 shows a graph showing photolysis emission spectra of NPET 2.

FIG. 67 shows a graph showing mass spectrum of compound 4.

FIG. 68 shows a graph showing mass spectrum of compound 5.

FIG. 69 shows a graph showing mass spectrum of compound 6.

FIG. 70 shows a graph showing mass spectrum of compound 7.

FIG. 71 shows a graph showing mass spectrum of NPET 1.

FIG. 72 shows a graph showing mass spectrum of NPET 2.

FIG. 73 shows a graph showing Fourier Transform Infrared Spectroscopy (FTIR) spectrum of 4.

FIG. 74 shows a graph showing FTIR spectrum of 5.

FIG. 75 shows a graph showing FTIR spectrum of NPET PPG 1.

FIG. 76 shows a graph showing FTIR spectrum of NPET PPG 2.

FIG. 77 shows a graph showing FT-IR spectral overlay of the uncleaved NPET 1 (bottom) and the photorelease products in CD₃CN (top) after irradiation with a 365 nm UV lamp (30-watt 4-core LED).

FIG. 78 shows a graph showing FT-IR spectral overlay of the uncleaved NPET 2 (bottom) and the photorelease products in CD₃CN (top) after irradiation with a 365 nm UV lamp (30-watt 4-core LED).

FIG. 79 shows a table showing Time-Dependent Density Functional Theory (TD-DFT) computed vertical excitation energies (VEEs) for the first three singlet excited states of reactants and products computed with PBE0/cc-pVDZ in acetonitrile PCM.

FIG. 80 shows a graph showing chromatograph and mass spectrum NPET 1 obtained by Liquid Chromatography-Mass Spectrometry (LCMS).

FIG. 81 shows a graph showing chromatograph and mass spectrum NPET 2 obtained by LCMS.

FIG. 82 shows molecular structure of NPET PPG scaffold.

FIG. 83 shows a synthetic route to synthesize NPETs 1 and 2. FIG. 83(A) shows reagents and conditions to synthesize hydroxylamine 7: (i) 1-(5-bromo-2-nitrophenyl)ethan-1-one, PdCl₂(PPh₃)₂, CuI, Et₃N, N₂, (85° C., 2 h, 79%); (ii) NaBH₄, 5:1 THF:MeOH, (−78° C.-rt, 15 min, 79%); (iii) N-hydroxyphthalimide, PPh₃, DIAD, THF, (−5° C.-rt, 14 h, 94%); (iv) NH₂NH₂·H₂O, MeOH (rt, 4 h, 81%). FIG. 83(B) shows reagents and conditions to synthesize NPETs 1 and 2 from hydroxylamine 7: (v) 4-ethylmorpholine, PyBOP, DMF (rt, 24 h, 58% and 87%, respectively).

FIG. 84(A) shows photolysis absorbance spectra for NPET 2 in 4:1 (v/v) MeCN:1M HCl after intervals of irradiation at 365 nm (concentration=10⁻⁶ M). FIG. 84(B) shows emission spectra for NPET 2 during and after irradiation at 365 nm showing an increase in fluorescence intensity (concentration=10⁻⁵ M).

FIG. 85 shows proposed mechanism for the cleavage of NPET PPGs.

FIG. 86 shows ¹H NMR overlay photolysis of NPET PPG 2 in CD₃CN:1M HCl (4:1) before (t=0 min) and after irradiation (t=8 mins) with a 365 nm UV lamp (30-watt 4-core LED). FIG. 85(A) shows ¹H-NMR cleavage trace from 8.5 ppm-5.00 ppm and FIG. 86(B) shows ¹H-NMR cleavage trace from 3.00 ppm-0.5 ppm. (NPET 2 concentration=5.14 mM).

FIG. 87 shows ¹H NMR spectral overlay (in CD₃CN, 400 MHz) of NPET 2, stability test over thirty days under ambient conditions.

FIG. 88 shows a table showing Time-Dependent Density Functional Theory (TD-DFT) computed vertical excitation energies (VEEs) for the first three singlet excited states computed with wB97X-D in acetonitrile phase change materials (PCM).

FIG. 89 shows representative natural transition orbitals (NTOs) of the first three excited states of the nitrosoketone product.

### DETAILED DESCRIPTION

Hydroxamic acids (HAs) are a class of organic compounds which have a carbonyl group (C═O) directly attached to a hydroxylamine (NH—OH) moiety. They were first discovered by German Chemist, Heinrich Lossen when he reacted hydroxylamine with diethyl oxalate to yield oxalohydroxamic acid, and this reaction was later identified by Wilhelm Lossen as the Lossen rearrangement reaction in 1869. HAs are structurally similar to amides. However, they are chemically different from them; amides are generally stable, whereas HAs are known to be very reactive.

Despite their high reactivity, HAs have extensively been applied in various fields of science due to their inherent bidentate metal-chelating abilities which allow them to bind or chelate transition metals like iron (Fe). In analytical chemistry, HAs are used to extract heavy metals from industrial wastewater systems. They form complexes with heavy metals and lead to distinct color changes which allows these metals to be identified and quantified upon extraction. In the polymer chemistry field, HAs form complexes with polymers such as polyethylene glycol to enhance contrast images in tumor imaging by complexing with the Fe metal in FeOₓ nanoparticles found in tumor imaging instruments. In organic chemistry, HAs are involved in multiple organic syntheses such as the Lossen rearrangement reaction for the formation of isocyanates which are used in various applications including tear gases, and for the preparation of 1,4,2-dioxazole protecting groups via reversible transketalization reactions. A major application of HAs in the medical field is their use as precursors of a variety of anti-cancer drugs. With their metal-chelating abilities, HAs bind to the active sites of histone deacetylases (HDACs), which are the enzymes responsible for causing cancers such as multiple myeloma and T-cell lymphoma. Although HAs have numerous applications in various fields of science, the traditional synthesis and purification of HAs have proven to be challenging.

Due to their high reactivity, traditional synthesis of HAs leads to the formation of poly-substituted byproducts, which make purification difficult. Some traditional HA syntheses involve toxic reagents such as KCN, which is toxic and leads to low HA yields. A final limitation to the synthesis and purification of HAs is the use of harsh reaction conditions such as acidic, basic, or oxidative, which can potentially degrade the protected HA.⁴⁵ An example is the Angeli-Rimini reaction which is the reaction of aldehydes with N-hydroxybenzenesulfonamide in the presence of a strong base, sodium methoxide, which forms an undesired byproduct, benzenesulfinic acid: a compound that self-decomposes at high temperatures.

To address the concerns above, scientists incorporate protecting groups (PGs) to temporarily mask the reactivity of HAs and other reactive functional groups. However, deprotection of conventional PGs involves harsh acidic, basic, and oxidative environment that tend to degrade the protected moiety. As a solution to this problem, photolabile protecting groups (PPGs) are utilized since only light, a less harsh approach is required to cleave desired compounds.

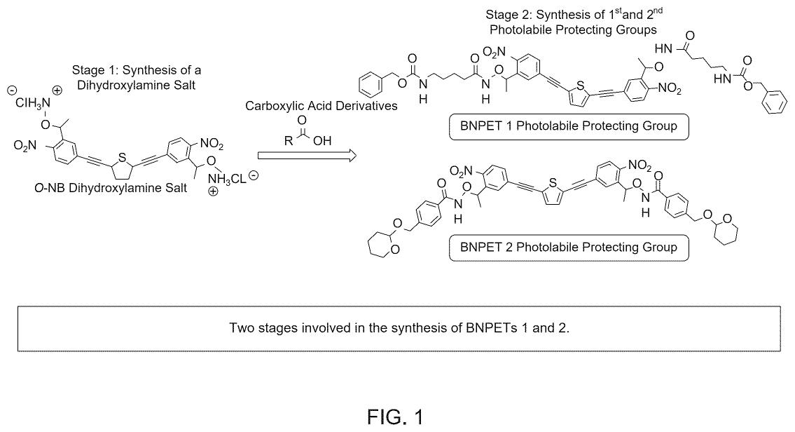

Described herein is synthesis, characterization, and application of the first and only thiophene-based visible light-absorbing, fluorescent-quenching ortho-nitrobenzyl (o-NB) PPGs (BNPETs 1 and 2) functionalized with different carboxylic acid derivatives to overcome the limitations of traditional HA synthesis and purification by cleaving HAs with visible light. Furthermore, the PPG scaffold is strategically designed with an electron-rich thiophene ring (pentagon with S in FIG. 1) and electron-poor nitro groups (O₂N and NO₂ in FIG. 1), to introduce an internal push-pull intramolecular charge transfer within the scaffold and red-shift the PPGs'absorption and emission wavelength to the visible region of the electromagnetic spectrum. To synthesize BNPETs 1 and 2, a stable dihydroxylamine salt, the precursor required for the synthesis of both PPGs, is synthesized via multi synthetic steps. Thereafter, the salt was coupled to different carboxylic acid derivatives (alkyl and phenyl) to yield BNPETs 1 and 2 (FIG. 1).

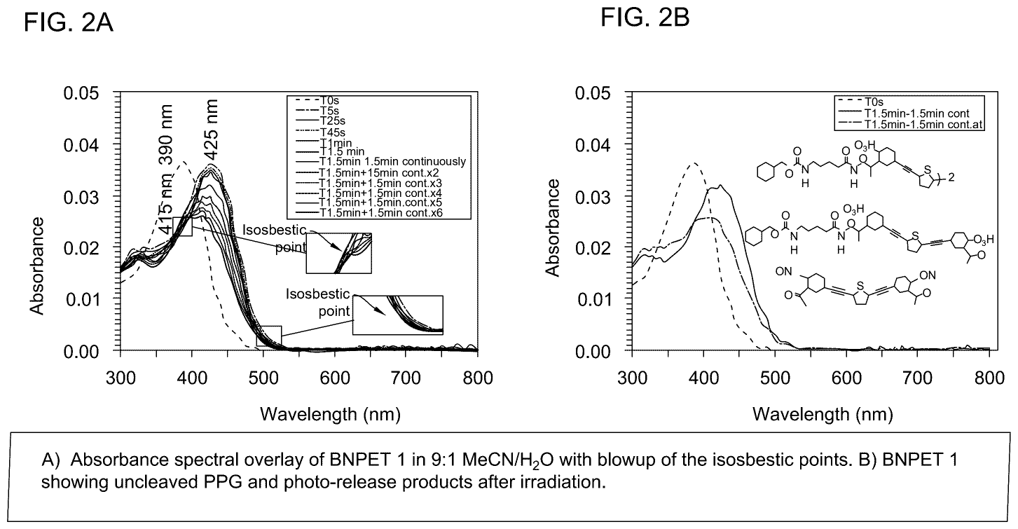

After synthesis and characterization of both BNPETs, photolysis experiments were conducted by dissolving both BNPETs 1 and 2 in 9:1 MeCN/H₂O and absorption and emission scans were taken before and after irradiation with visible light (395-405 nm LED lamp) to cleave HAs and produce a di-nitrosoketone byproduct which quenches the fluorescence and can be utilized to quantify HA release by monitoring the di-nitrosoketone byproduct's loss of absorption and emission. Before irradiation, the absorbance of BNPET 1 was measured at 390 nm (Nabs) which corresponds to the uncleaved BNPET 1. After irradiation for 5 seconds, a new red-shifted band was observed with λ_{abs} of 425 nm which corresponds to a mixture of the uncleaved PPG and one cleaved arm PPG. After 9 minutes of irradiation, a new blue-shifted absorbance measured at 415 nm (λ_{abs}) was observed which corresponds to the complete photolysis (FIGS. 2A and B). The same photolysis experiment was conducted for BNPET 2, and absorbance was measured before irradiation (t=0 seconds) at 385 nm (λ_{abs}). After irradiating the sample for 5 seconds, a new red-shifted absorbance band with λ_{abs} of 425 nm was observed which corresponds to a mixture of the uncleaved PPG and one cleaved arm PPG.

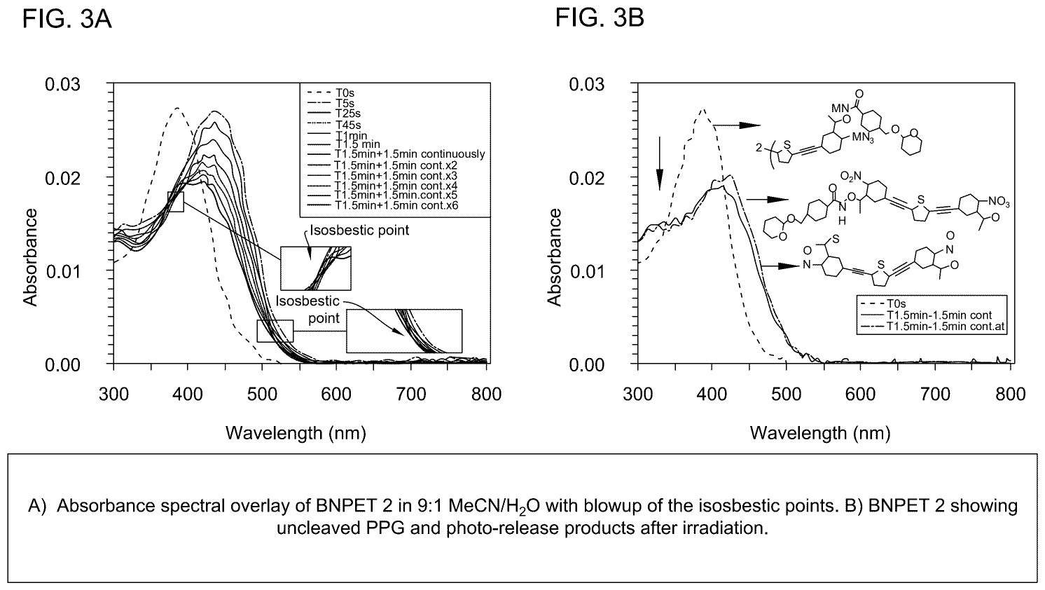

After 9 minutes of irradiation, a new blue-shifted band with λ_{abs} of 405 nm was observed, corresponding to full photolysis of BNPET 2 (FIGS. 3A and B). During the absorption photolysis experiment, two isosbestic points were observed which indicate clean photocleavage without any degradation. For the emission photolysis of BNPET 1, before irradiation, an emission band with λₑₘ of 628 nm was observed, corresponding to the uncleaved PPG.

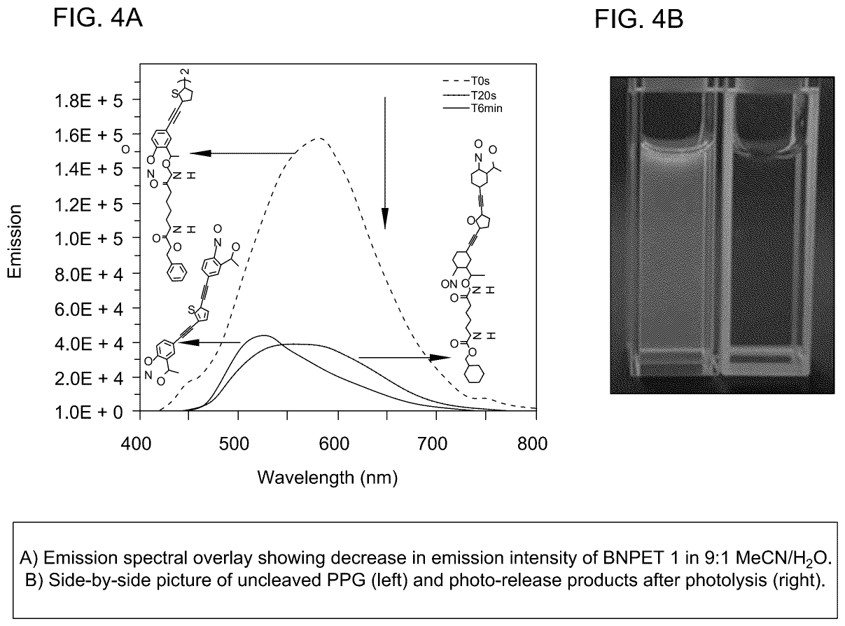

After 20 seconds of irradiation, a new red-shifted band (λₑₘ of 645 nm) with a significant decrease in emission intensity was observed, indicating photocleavage to form the PPG with one cleaved arm. After 4.5 minutes of irradiation, a blue-shifted band with λₑₘ of 539 nm was observed, which corresponds to the complete photocleavage of both arms of the PPG (FIG. 4A). After photolysis, a side-by-side picture of the uncleaved PPG and the irradiated sample was taken under UV light to compare the quenching effect which is caused by photoinduced electron transfer (PET) (FIG. 4B). The same photolysis experiment was conducted on BNPET 2, and a similar trend was observed. Before irradiation, an emission band with a λₑₘ of 618 nm was observed, and after 5 seconds of irradiation, a new blue-shifted emission band with decreased intensity was observed at 592 nm (λₑₘ).

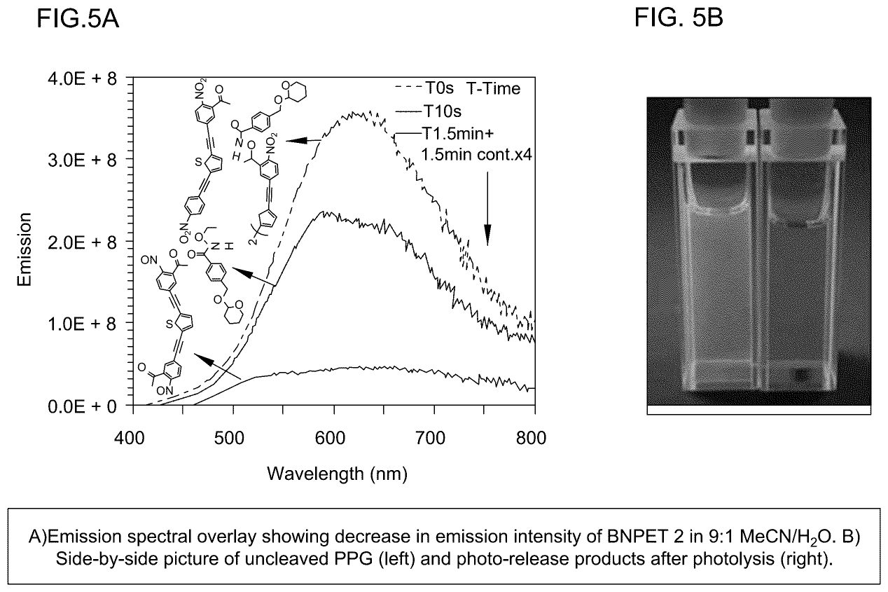

The sample was irradiated for 4.5 minutes and a new red-shifted emission band with quenched fluorescence (λₑₘ=661 nm) was observed, which corresponds to the full photolysis of BNPET 2 as illustrated in FIGS. 5A and B.

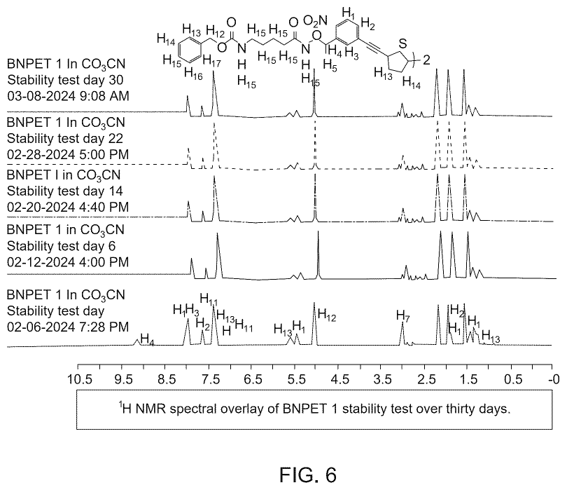

Finally, stability tests were conducted on both PPGs via ¹H NMR experiments to investigate the shelf lives of both BNPETs. For BNPET 1, it was observed that after thirty days of conducting the experiment, all proton signals were still the same based on the ¹H NMR taken (FIG. 6). The stability of BNPET 2 was tested using the same experimental protocol for BNPET 1. After fourteen days, minor degradation was observed, however, the PPG was still intact.

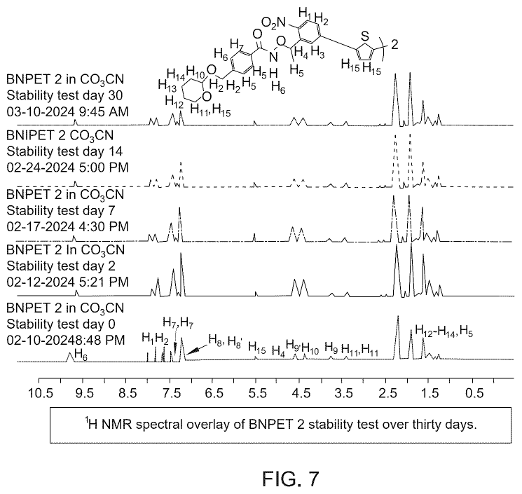

After thirty days of testing, degradation of BNPET 2 was still minor and all proton peaks were identifiable on the ¹H NMR spectra as illustrated in FIG. 7. Based on the results obtained, it was confirmed that both BNPETs 1 and 2 have a shelf life of more than 30 days.

Described herein is the synthesis, characterization, and application of two new visible light-absorbing, fluorescent-quenching thiophene-based o-NB PPGs, BNPETs 1 and 2, for the synthesis of HAs as a solution to the problems associated with traditional HA synthesis and purification. Photolysis of both PPGs leads to the formation of a di-nitrosoketone byproduct which quenches the fluorescence and can be used to quantify the amount of HAs cleaved by measuring the loss of emission and absorption. Findings from this research will be used in the design of future fluorescent-quenching PPGs for light regulation of biomolecules and cleavage of other reactive functional groups.

Hydroxamic acids (HAS), a class of organic compounds bearing a carbonyl group (C═O) directly connected to a hydroxylamine (HN—OH) moiety, have been the subject of extensive research due to its versatile applications such as being used as metal chelators in the removal of toxic metals from seawater and as precursors of several anti-cancer drugs.

Nevertheless, synthesis of HAs is widely known to be problematic and the purification of the final HA product can be challenging because HAs are extremely reactive and will often form a mixture of poly-substituted by-products. Difficulties in synthesizing and purifying these types of compounds are routinely circumvented by adding protecting groups (PGs) to temporarily inactivate the reactivity of a functional group. However, the PGs are traditionally removed using harsh reaction conditions such as acidic, oxidative, or basic, which presents an issue because harsh reaction conditions can potentially degrade the protected molecule. To remedy this, chemists will often use photolabile protecting groups (PPGs) because they require only light to remove the PG, which is a less harsh condition.

The first example of PPGs, the ortho-nitrobenzyl (o-NB) series, were synthesized in 1966 by Barltrop et al. as a more desirable approach to tackle the issue of potential degradation of the protected molecule during deprotection of conventional PGs using nitrobenzyl-compounds containing a C-H bond in the ortho-position. However, Barltrop and coworkers observed that the nitroso benzaldehyde by-product formed after PPG deprotection undergoes a side reaction with amine release products resulting in low yields of the released products. To try to solve this problem, chemists added a methyl (—CH₃) group at the benzylic position ortho to the nitro group producing a less reactive nitrosoketone by-product instead of nitroso benzaldehyde upon irradiation resulting in new PPGs with significantly increased yields of the released products. Since then, investigation of the applications of o-NB PPGs has increased rapidly; however, there are limited examples of PPG systems with a fluorescent by-product upon photorelease.

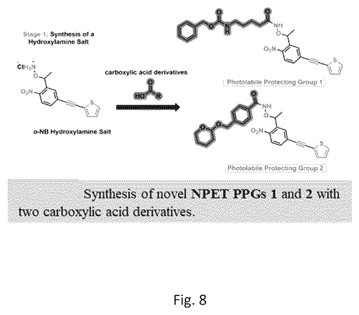

Described herein is the first and only example of a pro-fluorescent thiophene-based o-NB PPG for the synthesis of HAs in high yield with an easily detectable fluorescent by-product that can be used to quantify the amount of HA produced. Described herein is the design of two novel thiophene-based o-NB PPGs with similar structures and functionalized with different R groups (alkyl and phenyl referred to as NPET 1 and 2, respectively) were synthesized as a more attractive approach to overcoming the problems associated with HA synthesis and their purification (FIG. 8).

Synthesis of these NPET PPGs was conducted in two stages: synthesis of the PPG scaffold containing a hydroxylamine salt and then reacting this salt with two carboxylic acids derivatives to produce NPETs 1 and 2 (FIG. 8). An aromatic heterocyclic core was incorporated into the NPET PPG design to ensure a highly conjugated z-system structure. An internal push-pull character that causes intramolecular charge transfer throughout the system throughout the PPG scaffold was introduced to ensure the PPGs possess red-shifted emission and absorbance wavelengths. To achieve this, a —NO₂ group (O₂N in FIG. 8) was chosen as an electron withdrawing group at the para-position to an electron rich thiophene heterocycle chosen as the electron donating group (pentagon with S in FIG. 8 to induce the push-pull effect throughout the conjugated z-system. To guarantee the NPET PPG scaffold possessed a highly conjugated π-system with a coplanar geometry, a rigid core was needed to greatly enhance the desired push-pull effect throughout the PPG while keeping the p-orbitals overlapping. Thus, an alkyne linker (each O connected to NH and the O connected to ClH₃N in FIG. 8) was used to bridge the electronic groups.

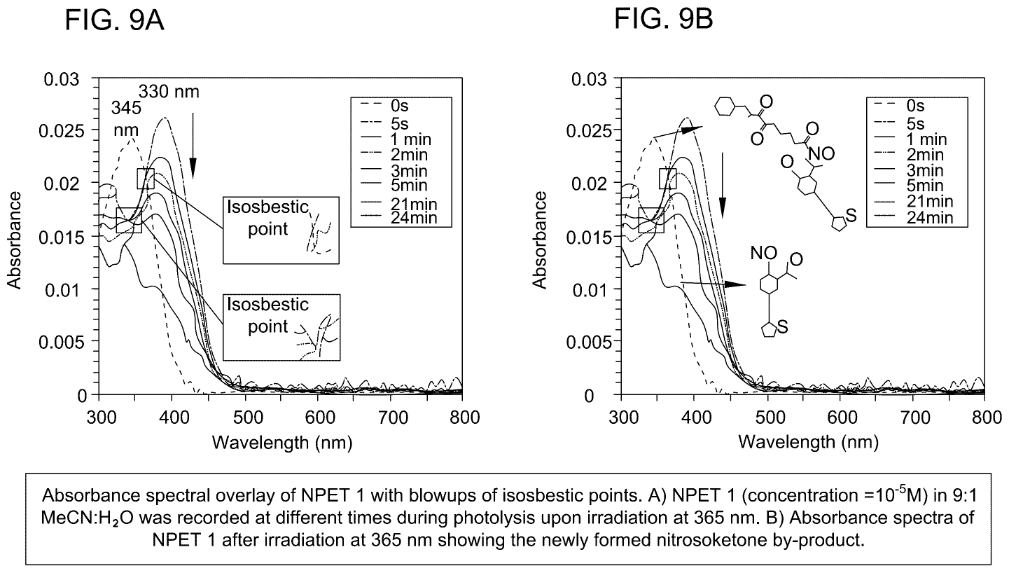

After successfully synthesizing and characterizing all precursors and NPETs 1 and 2, the photolytic release properties of NPETs 1 and 2 were examined by irradiating prepared samples of the two PPGs in a MeCN:H₂O solvent system and observing the change in absorbance and fluorescence wavelengths over time via UV-Vis and emission spectroscopy. An absorbance scan was taken before irradiation (time=0 sec) with a λₘₐₓ of 345 nm (FIG. 9A). This corresponds to the uncleaved NPET 1. The sample was then irradiated with a 30 watt 4-core LED 365 nm UV lamp for 5 sec, followed by 1 min intervals for 24 min with the λₘₐₓ and relevant absorbance recorded. After 5 sec, a new red-shifted absorbance band (390 nm) was observed which corresponds to a new species being formed due to photocleavage of the NPET 1, a nitrosoketone by-product and hydroxamic acid product. A gradual decrease in absorbance intensity was observed until it remained constant after 24 min of irradiation indicating that the HA moiety, benzyl (5-(hydroxyamino)-5-oxopentyl)carbamate, is now completely cleaved from the NPET 1, which corresponds to the final nitrosoketone by-product (FIG. 9B). Furthermore, the presence of two isosbestic points at 365 nm and 340 nm observed in the spectral overlay indicates that a clean photochemical reaction without degradation occurred leading to stable release products. The same experiment was conducted to cleave N-hydroxy-4-(((tetrahydro-2H-pyran-2-yl)oxy)methyl)benzamide from NPET 2.

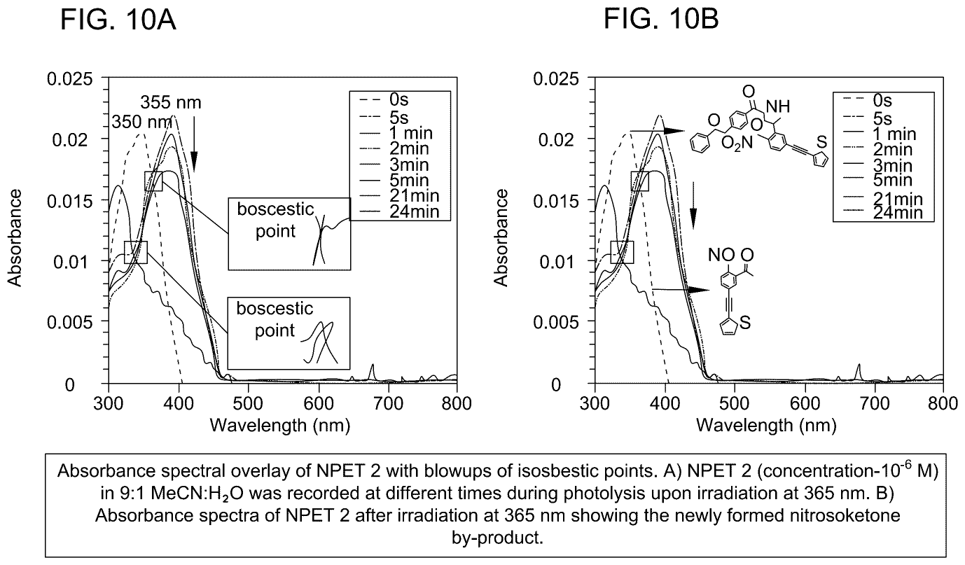

An absorbance scan was taken before irradiation (time=0 sec) with a λₘₐₓ of 350 nm (FIG. 10A). This corresponds to the uncleaved NPET 2. The sample was then irradiated with a 30 watt 4-core LED 365 nm UV lamp at 5 sec intervals for 90 sec, followed by a continuous 90 sec irradiation for (total time=34 min 50 sec), and an absorbance scan was taken after each irradiation with the wavelength maxima recorded. After 5 sec, a new red-shifted absorbance band (395 nm) was observed which corresponds to a new species being formed due to photocleavage of N-hydroxy-4-(((tetrahydro-2H-pyran-2-yl)oxy)methyl)benzamide from NPET 2, resulting in a nitrosoketone by-product. A gradual decrease in absorbance intensity was observed until remaining constant after 34 min 50 sec indicating that N-hydroxy-4-(((tetrahydro-2H-pyran-2-yl)oxy)methyl)benzamide is now completely cleaved from the NPET 2, which corresponds to the final nitrosoketone product (FIG. 10B). Furthermore, as mentioned previously, the presence of two isosbestic points observed in the spectral overlay indicates that a clean photochemical reaction occurred leading to stable release products.

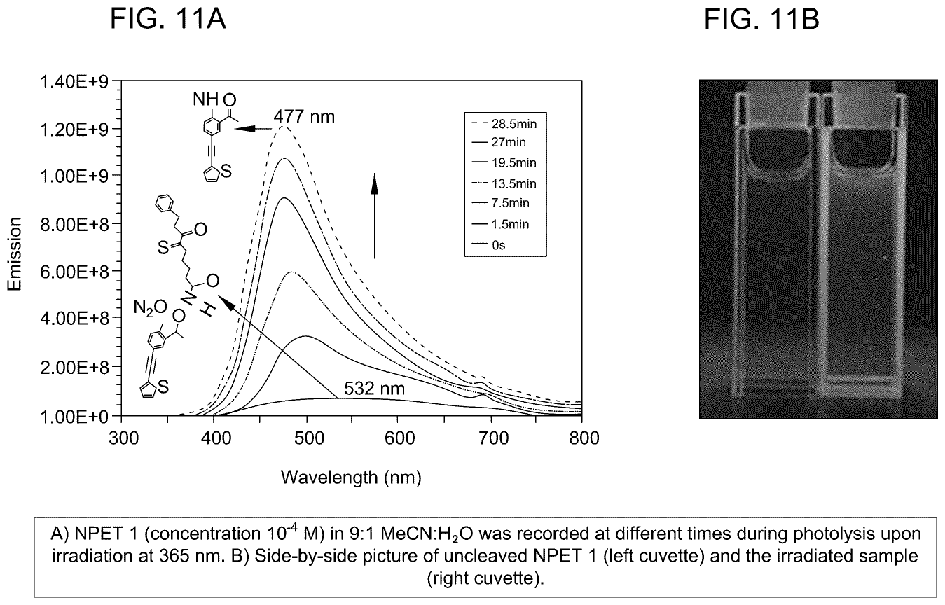

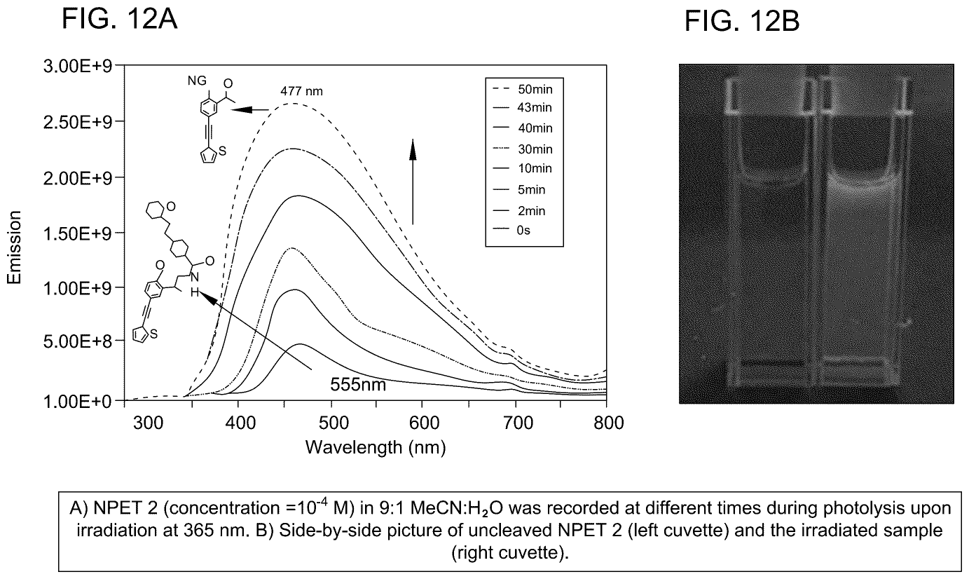

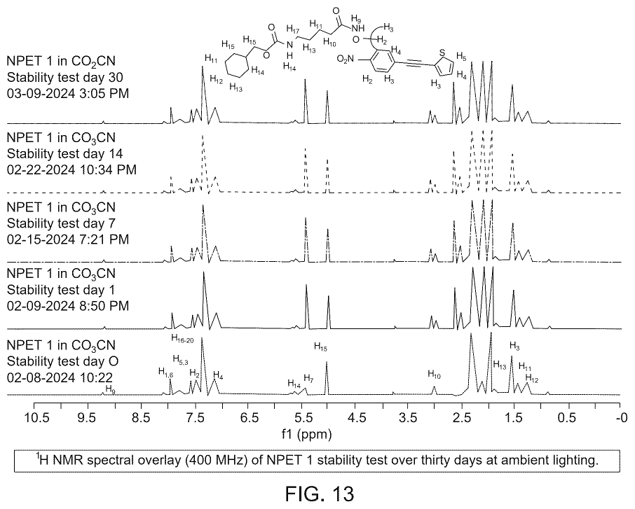

As hypothesized, NPET 1 and 2 are weakly emissive and the emission cannot be seen with the naked eye before irradiation, however, NPET 1 was fully cleaved after 28.50 min with a 17-fold increase in fluorescent intensity (FIG. 11), while NPET 2 was fully cleaved after 50 min with a 40-fold increase in fluorescent intensity (FIG. 12). This indicated that the new strongly emissive species (the nitrosoketone by-product) was produced resulting in a highly fluorescent sample that can be detected with the naked eye. After completion of the photolysis studies of NPETs 1 and 2, the stability of both PPGs was studied by evaluating their shelf lives by conducting ¹H NMR time course experiments. A ¹H NMR was taken daily to monitor the stability of both PPGs. Minor degradation was observed in the NPET 1 sample after day one (FIG. 13).

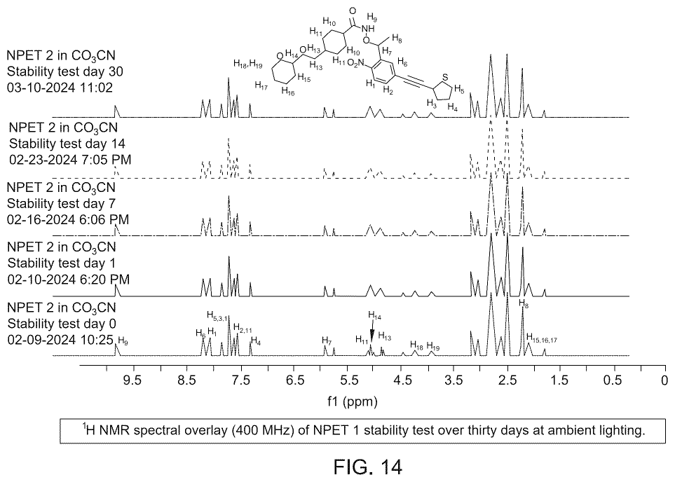

After thirty days in ambient temperature, the ¹H NMR and corresponding proton peaks observed can be identified as the initial PPG indicating that NPET 1 is stable with minor degradation. The same experiment was conducted to test for the stability of NPET 2.After seven day, minor degradation was observed, however, the PPG was still intact (FIG. 14). After thirty days of testing, degradation of NPET 2 was still minor, and all proton peaks were identifiable on the ¹H NMR spectra indicating that NPET 2 is also stable.

Described herein is the synthesis, characterization, and application of new pro-fluorescent thiophene-based o-NB PPGs for the synthesis of HAs as a solution to the limitations of HA synthesis and purification by releasing HAs with an easily detectable nitroso-ketone diagnostic fluorescent by-product, upon irradiation with light. Lastly, these results will aid in the development of PPGs capable to photo-release various hard to synthesize functional groups like carboxylic acids, sulfonates, and phosphates because of its appealing properties like strong molar absorption coefficients and fluorescent properties at longer wavelengths that allow monitoring of the reaction course.

### General Information

Starting materials were produced in-house and solvents were purchased from commercial suppliers unless noted otherwise. Materials obtained from commercial suppliers were used without further purification. 1-(5-Bromo-2-nitrophenyl)ethanone was purchased from ChemScene. p-Toluenesulfonic acid was purchased from Alfa Aesar. Bis(triphenylphosphine)palladium(II) dichloride, sodium borohydride, N-Hydroxyphthalimide and 2-ethynyl thiophene were purchased from Sigma Aldrich. Copper iodide and hydrazine hydrate were purchased from Acros Organics. Triethylamine and methanol were purchased from VWR International. Potassium carbonate was purchased from Flinn Scientific, Inc. Triphenylphosphine, diisopropyl azodicarboxylate, THF, and hydrochloric acid were purchased from Thermo Fisher Scientific. The NMR solvents, CDCl₃ was purchased from Cambridge Isotope Laboratories, acetonitrile-do and DMSO-d₆ were purchased from Thermo Fisher Scientific.

Organic solutions were concentrated by rotary evaporation at ca. 12 Torr. ¹H NMR spectra were recorded at 400 MHz and ¹³C NMR spectra at 100 MHz on a Bruker 400'54 Ascend spectrometer; chemical shifts are expressed in parts per million (δ scale) downfield from tetramethylsilane (δ=0.00) and are referenced to residual protium in the NMR solvent (CDCl₃: δ=7.26, CD₃CN: δ=1.96, DMSO-d₆: δ=2.54) for ¹H NMR and relative to the central CDCl₃ (δ=77.16), CD₃CN (δ=118.26) and DMSO-d₆ (δ=39.52) for ¹³C NMR. Data are presented as follows: chemical shift, multiplicity (s=singlet, d=doublet, t=triplet, q=quartet, quint=quintet, m=multiplet and/or multiple resonances), coupling constant in Hertz (Hz), integration. FTIR spectra were recorded on a PerkinElmer Spectrum 100 FTIR spectrometer. All samples were placed over the detector and crushed using the anvil attached on the Perkin Elmer Spectrum 100 FTIR spectrometer featuring an attenuated total reflection (ATR) sampler equipped with a diamond crystal. Spectra were obtained in the range of 600-4000 cm⁻¹.

UV-Vis Optical spectra were recorded on a Cary 60 Spectrophotometer (Agilent Technologies, Inc.). Spectra were obtained in the range of 200-800 nm in a 1 cm quartz cuvette. Emission spectra were recorded on a HORIBA Fluorolog QM-75-11-C Spectrofluorometer. Melting points were recorded on a Mpa161 Digimelt Melting Point apparatus from Stanford Research Systems. Palladium cross-coupling reactions were performed under a nitrogen atmosphere in glassware that had been oven-dried for 24 h. All reactions and manipulations involving air-sensitive compounds were performed using standard Schlenk techniques. N₂ gas was purchased from Airgas. Thin-layer chromatography was performed on TLC silica gel 60 F₂₅₄ aluminum sheets from Sorbtech. Flash column chromatography was performed using a CombiFlash® Rf from Teledyne ISCO.

### Mass Spectrometry and LCMS Protocols

Mass spectrometry experiments were conducted with LTQ XL mass spectrometer (Thermo Fisher Scientific, Waltham, MA) equipped with DART source (IonSense, Saugus, MA). DART source was optimized by varying gas heater temperatures (200-400° C.) and setting the helium gas flow rate at 2.0 mL/s. The DART mass spectra were acquired in the range of m/z 100-2000.

To assess the purity of the final compounds (NPET 1 and NPET 2), liquid chromatography coupled with mass spectrometry was conducted using Vanquish Flex HPLC coupled with Orbitrap Exploris™ 240 Mass Spectrometer (Thermo Fisher Scientific, Waltham, MA). Compounds samples were prepared at 500 μM concentration using 10% acetonitrile containing 0.1% formic acid. A C18 reverse-phase column (Acclaim™) was employed using a 15-min long gradient of water (solvent A) and acetonitrile (solvent B) containing 0.1% formic acid. Gradient was set at 0 to 50% B in 4 mins, 50%-98% in 4-13 mins and 0% in 14 mins. For mass spectrometry experiments, HESI (heated electrospray ionization) source was set in positive mode, spray voltage fixed at 3900 volts, Sheath and Auxiliary Gas was set at 35 and 7, Ion Transfer Tube and Vaporize temperatures were set at 320° C. and 275° C. respectively. The ESI mass spectra were acquired in the range of m/z 100-1500 with resolution of 60K and RF lens voltage at 50%.

The NPET 1 compound was eluted at 11.06 min with a very tense single peak which confirmed that the compound is highly pure (˜95%). The corresponding mass spectrum also showed a peak at m/z 522.16547 exactly matched with the theoretical mass of the compound. A dimer peak is also noticed at m/z 1043.32. Similarly, the NPET 2 compound was eluted at 11.38 min with a single strong peak exhibited the high purity of the compound. A peak identified at m/z 507.15217 corresponds to the mass of the compound NPET 2.

### Specifications of the UV Lamp Used for Photolysis

The photolysis experiments were conducted with a high power 30 W 4-core LED 365 nm UV LUXNOVAQ lamp (equipped with black filter lens) powered by 300 mAh rechargeable lithium polymer batteries with a brightness of 200 lumen.

### UV-Vis and Emission Photolysis Procedure for NPETs 1 and 2

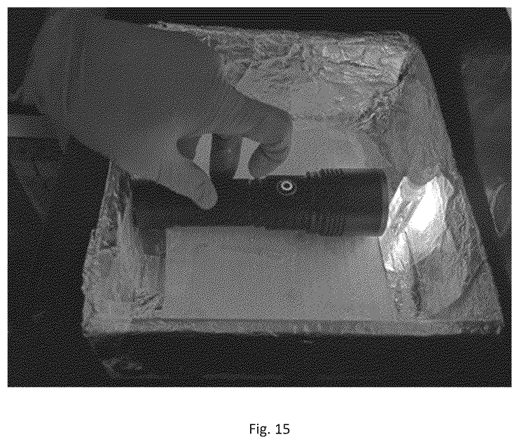

The sample-containing cuvette was irradiated using a 365 nm UV lamp (30-watt, 4-core LED) within an aluminum-enclosed casing as shown in FIG. 15. The cuvette was positioned 2 cm away from the light source for UV-Vis and emission studies.

### ¹H-NMR Photolysis Procedure for NPETs 1 and 2

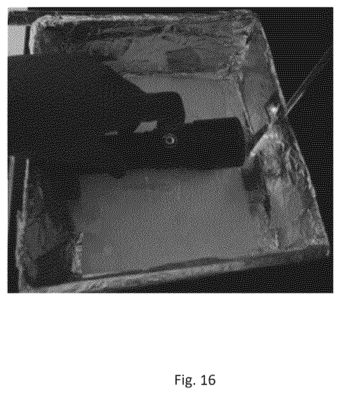

The samples were prepared by dissolving ˜1 mg of NPETs 1 or 2 in 0.5 mL of deuterated acetonitrile (CD₃CN). A borosilicate NMR tube containing the sample was irradiated with a 365 nm UV lamp (30-watt 4-core LED) in an aluminum-surrounded casing as shown in FIG. 16. The NMR tube was placed at a distance of 2 cm of the light source to study photolysis behavior. NMR spectra were acquired using a 400 MHz Bruker NMR with 64 scans.

### ¹H-NMR Stability Testing Procedure for NPETs 1 and 2

The samples were prepared by dissolving ˜10 mg of NPET 1 or 2 in 0.5 mL of deuterated CD₃CN. The solution was transferred to a clean borosilicate NMR tube and left on the benchtop for 30 days. Daily ¹H-NMR spectra were recorded to monitor the stability of PPG. NMR spectra were acquired using a 400 MHz Bruker NMR instrument with 16 scans.

### Synthesis Protocols

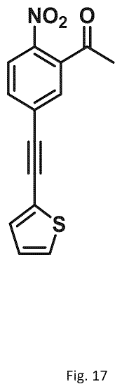

FIG. 17 shows a representation of 1-(2-nitro-5-(thiophen-2-ylethynyl)phenyl)ethan-1-one (4).

1-(5-Bromo-2-nitrophenyl)ethanone (0.50 g, 2.06 mmol, 1.00 equiv.), PdCl₂ (PPh₃)₂ (0.02 g, 0.02 mmol, 1 mol %), and CuI (0.01 g, 0.02 mmol, 1 mol %) and a magnetic stir bar were added to a 50 mL Schlenk flask dried overnight in an oven. The Schlenk flask was sealed, evacuated overnight, and filled with N₂, followed by the addition of 2-ethynyl thiophene 3 (0.25 mL, 2.5 mmol, 1.2 equiv.) in Et₃N (20 mL, purged with N₂ with stirring for 1 h) resulting in a brown mixture. The reaction mixture was allowed to vigorously stir under N₂ atmosphere at 85° C. After 2 h, the reaction was complete as determined with TLC and ¹H NMR. The solvent was evaporated under vacuo leaving a crude solid. The crude material was purified by silica gel flash column chromatography using a combi-flash with a (0-5.6% EA) gradient. The desired fraction tubes were collected and the solvent was evaporated in vacuo to yield 4 as a yellow solid (0.45 g, 3.57 mmol, 79% yield). R_{f}=0.30 (Hex:EA 7:1). M.p.: 130-135° C. ¹H NMR (400 MHz, CDCl₃, 25° C., δ): 8.10 (d, J=8.5 Hz, 1H), 7.66 (dd, J=1.7, 8.5 Hz, 1H), 7.50 (d, J=1.7 Hz, 1H), 7.39 (d, J=3.6 Hz, 2H), 7.07 (t, J=3.8 Hz, 1H), 2.57 (s, 3H). ¹³C {¹H} NMR (100 MHz, CDCl₃, 25° C., δ): 199.0, 144.1, 138.5, 133.7, 132.6, 130.0, 129.6, 129.3, 127.5, 124.7, 121.6, 90.6, 89.2, 30.3; FTIR (ATR) (cm⁻¹): 3101 (w), 3070 (w), 2918 (w), 2837 (w), 2190 (m), 1816 (w), 1702 (m), 1577 (m), 1509 (s), 1416 (w), 1389 (w), 1328 (s), 1260 (m), 1232 (s), 1207 (s), 1105 (m), 1040 (m), 966 (w), 887 (m), 828 (s), 759 (w), 718 (s), 692 (s), 635 (m). HRMS (DART-MS) m/z: [M+H]⁺ Calcd for C₁₄H₉NO₃SH 272.0381; found 272.0333.

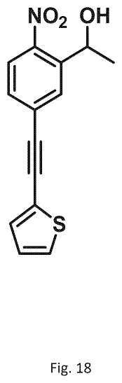

FIG. 18 shows a representation of 1-(2-nitro-5-(thiophen-2-ylethynyl)phenyl)ethan-1-ol (5).

Compound 4 (0.3 g, 1.11 mmol, 1.0 equiv.) was added to a 50 mL round bottom flask and dissolved in THF/MeOH (5:1; 15 mL:3 mL), and the mixture was cooled to −78° C. in a dry ice/acetone bath for 30 min. NaBH₄ (0.04 g, 1.13 mmol, 1.02 equiv.) was added in small portions over 10 minutes, and the reaction was stirred for 15 minutes while allowed to reach room temperature. TLC was taken to confirm the completion of the reaction, and the reaction was concentrated under reduced pressure. Once concentrated, DI H₂O was added and the organic layer was extracted with EA (4×30 mL). The organic layer was washed with brine solution (1×20 mL), dried over anhydrous MgSO₄, gravity-filtered, and concentrated under reduced pressure. The crude product was purified by silica gel flash column chromatography using a combi-flash with 100% hexanes to remove impurities before alternating to 0-5% EA gradient. The desired fraction tubes were collected, and the solvent was evaporated in vacuo to yield 5 as a yellow solid (0.24 g, 0.87 mmol, 79% yield). R_{f}=0.57 (Hex:EA 3:1). M.p.: 112-117° C. ¹H NMR (400 MHz, CDCl₃, 25° C., δ): 7.97 (d, J=1.7 Hz, 1H), 7.93 (d, J=8.5 Hz, 1H), 7.51 (dd, J=1.8, 8.5 Hz, 1H), 7.37 (d, J=3.7 Hz, 2H), 7.05 (t, J=3.7 Hz, 1H), 5.48 (q, J=6.4 Hz, 1H), 2.32 (s, 1H), 1.59 (d, J=6.4 Hz, 3H). ¹³C {1H} NMR (100 MHz, CDCl₃, 25° C., δ): 146.5, 141.6, 133.2, 130.5, 130.5, 129.1, 128.7, 127.4, 124.8, 122.1, 91.4, 87.3, 65.6, 24.3; FTIR (ATR) (cm⁻¹): 3331 (w), 3106 (w), 2977 (w), 2292 (w), 2198 (m), 1909 (w), 1810 (w), 1724 (w), 1706 (m), 1489 (w), 1435 (w), 1323 (s), 1288 (s), 1216 (w), 1168 (m), 1093 (s), 884 (w), 830 (s), 710 (s). HRMS (DART-MS) m/z: [M−OH]⁺ Calcd for C₁₄H₁₀NO₂S 256.0390; found 256.0000. As there is no protonation site available in this compound 5, during DART ionization, OH has been removed and created a carbocation, which showed higher stability than the parent compound.

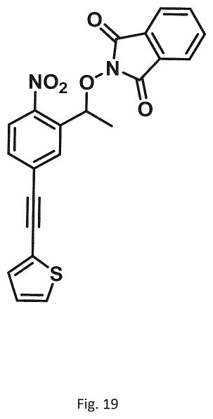

FIG. 19 shows a representation of 2-(1-(2-nitro-5-(thiophen-2-ylethynyl)phenyl)ethoxy)isoindoline-1,3-dione (6).

Compound 5 (0.45 g, 1.65 mmol, 1.0 equiv.) was added in a 25 mL round bottom flask and dissolved in THF (5 mL). The mixture was allowed to stir at −5° C. in an ice-brine bath for 10 min. N-hydroxyphthalimide (0.30 g, 1.82 mmol, 1.1 equiv.), triphenylphosphine (0.65 g, 2.49 mmol, 1.5 equiv.), and diisopropyl azodicarboxylate (0.49 mL, 2.48 mmol, 1.5 equiv.) were added and the reaction mixture was stirred while allowed to reach room temperature for 14 h, as the reaction completion was determine by TLC and ¹H NMR. The solvent was evaporated under vacuo and the crude product was purified by silica gel flash column chromatography using a combiflash with 100% hexanes for 10 min to remove impurities before alternating from 0-40% DCM gradient. The desired fraction tubes were collected and the solvent was evaporated in vacuo yielding 6 as a yellow solid (0.62 g, 1.56 mmol, 94% yield). R_{f}=0.39 (Hex:EA 3:1). mp 166-169° C. ¹H NMR (400 MHz, CDCl₃, 25° C., δ): 8.30 (d, J=1.5 Hz, 1H), 7.93 (d, J=8.5 Hz, 1H), 7.80 (d, J=5.4 Hz, 2H), 7.74 (d, J=5.4 Hz, 2H), 7.56 (dd, J=1.6, 8.5 Hz, 1H), 7.40 (d, J=3.7 Hz, 2H), 7.07 (t, J=3.7 Hz, 1H), 5.99 (q, J=6.4 Hz, 1H), 1.82 (d, J=6.4 Hz, 3H). ¹³C {¹H} NMR (100 MHz, CDCl₃, 25° C., δ): 163.6, 146.6, 136.7, 134.6, 133.3, 131.7, 131.4, 129.1, 128.8, 128.7, 127.4, 124.5, 123.7, 122.2, 91.3, 87.8, 80.6, 21.7. HRMS (DART-MS) m/z: [M+H]⁺ Calcd for C₂₂H₁₄N₂O₅SH 419.0701; found 418.9967.

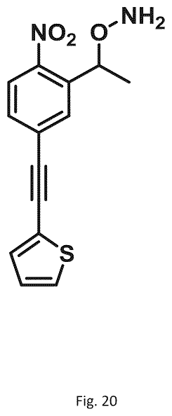

FIG. 20 shows a representation of O-(1-(2-nitro-5-(thiophen-2-ylethynyl)phenyl)ethyl)hydroxylamine (7).

Working in the dark as much as possible, compound 6 (0.18 g, 0.43 mmol, 1.0 equiv.) was added in a 25 mL round bottom flask and dissolved in MeOH (5 mL). Hydrazine hydrate (64% v/v in water) (0.06 mL, 1.29 mmol, 3.0 eq) was added dropwise, and the mixture was stirred at room temperature for 4 h, as the reaction completion was determined by TLC and ¹H NMR. The reaction mixture was then vacuum filtered through celite, washed with Et₂O, gravity filtered twice, and concentrated under reduced pressure to yield 7 as a yellow liquid (0.10 g, 0.35 mmol, 81% yield). R_{f}=0.70 (Hex:EA 3:1). ¹H NMR (400 MHz, CDCl₃) δ 7.96 (d, J=8.5 Hz, 1H), 7.82 (d, J=1.9 Hz, 1H), 7.52 (dd, J=8.4, 1.9 Hz, 1H), 7.41-7.35 (m, 2H), 7.06 (dd, J=5.2, 3.8 Hz, 1H), 5.37 (s, 2H), 5.27 (q, J=6.4 Hz, 1H), 1.51 (d, J=6.4 Hz, 3H). ¹³C {¹H} NMR (100 MHz, CDCl₃, 25° C., δ): 147.5, 140.6, 133.1, 130.4, 130.1, 128.9, 128.7, 127.4, 124.9, 122.1, 91.5, 87.1, 78.2, 21.4. HRMS (DART-MS) m/z: [M+H]⁺ Calcd for C₁₄H₁₂N₂O₃SH 289.0647; found 289.0462.

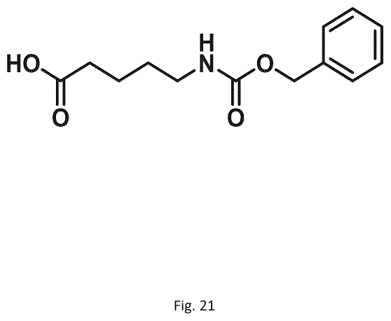

FIG. 21 shows a representation of 5-(((benzyloxy)carbonyl)amino)pentanoic acid (8).

5-aminovaleric acid (2.00 g, 17.1 mmol. 1.0 equiv.) was added to a 50 mL round bottom flask and dissolved in aqueous NaOH (2 M, 17.1 mL) while stirring slowly in an ice bath. CbzCl (2.23 mL, 17.1 mmol, 1.0 equiv.) was added dropwise at 0° C. and stirred overnight while reaching room temperature. The reaction mixture was extracted with Et₂O (3×25 mL), and the aqueous layer was acidified by the addition of aqueous HCl (1 M, 14 mL) while stirring for 5 min. The reaction mixture was placed in an ice bath for 30 min and the colorless precipitate was collected via vacuum filtration, washed with cold DI water, and dried to yield 8 as a colorless solid (3.88 g, 15.43 mmol, 90%). R_{f}=0.31 (Hex:EA 1:1). M.p.: 105-107.7° C. ¹H NMR (400 MHz, DMSO-d₆, 25° C., δ): 7.38-7.23 (m, 6H), 5.00 (s, 2H), 2.98 (t, J=6.0 Hz, 2H), 2.18 (t, J=7.4 Hz, 2H), 1.50-1.38 (m, 4H). ¹³C {¹H} NMR (100 MHz, DMSO-d₆, 25° C., δ): 174.3, 156.0, 137.2, 128.2, 127.6, 127.6, 65.0, 33.2, 28.8, 21.7.

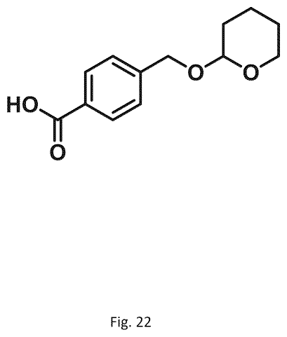

FIG. 22 shows a representation of 4-(((tetrahydro-2H-pyran-2-yl)oxy)methyl)benzoic acid (9).

P-toluenesulfonic acid monohydrate (0.03 g, 0.13 mmol. 0.01 equiv.) and 4-(hydroxymethyl)benzoic acid (2.00 g, 13.15 mmol. 1.0 equiv.) were placed into a 50 mL round bottom flask and dissolved in THF (15 mL). The reaction mixture was stirred at room temperature for 15 min, followed by the addition of 3,4-dihydro-2H-pyran (2 mL, 23.35 mmol, 1.7 equiv.). The mixture was then stirred vigorously while refluxing for 13 h, as the completion of the reaction was confirmed by TLC and ¹H NMR. The crude compound was purified by silica gel flash column chromatography using a combiflash with 100% hexanes for 3 min to remove impurities before alternating from 0-50% EA gradient. The desired fraction tubes were collected and the solvent was evaporated in vacuo to yield 9 as a colorless powder (2.83 g, 11.99 mmol, 91%). R_{f}=0.59 (Hex:EA 1:1). M.p.: 91-93.2° C. ¹H NMR (300 MHz, CDCl₃, 25° C., δ): 8.09 (d, J=8.2 Hz, 2H), 7.47 (d, J=8.2 Hz, 2H), 4.87 (d, J=13.04 Hz, 1H), 4.73 (t, J=3.4 Hz, 1H), 4.59 (d, J=13.03 Hz, 1H), 3.95-3.87 (m, 1H), 3.60-3.54 (m, 1H), 1.92-1.55 (m, 6H). ¹³C {¹H} NMR (75 MHz, CDCl₃, 25° C., δ): 170.6, 144.7, 130.3, 127.3, 98.1, 68.2, 62.2, 30.5, 25.4, 19.3.

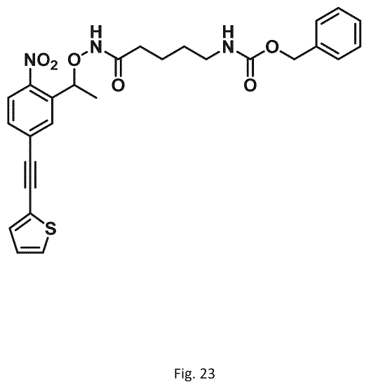

FIG. 23 shows a representation of benzyl (5-((1-(2-nitro-5-(thiophen-2-ylethynyl) phenyl) ethoxy) amino)-5-oxopentyl) carbamate (NPET 1).

Working in the dark as much as possible, compound 8 (0.10 g, 0.40 mmol, 1.2 equiv.) was placed in a 25 mL round bottom flask and dissolved in DMF (5 mL). PyBOP (0.22 g, 0.43 mmol, 1.3 equiv.) and 4-ethylmorpholine (0.25 mL, 1.98 mmol, 6.0 equiv.) were added while stirring for 20 min at room temperature, resulting in a color change from colorless to pink. Compound 7 (0.10 g, 0.33 mmol, 1.0 equiv.) was added to the reaction mixture, resulting in an orange color change. The reaction mixture was allowed to stir overnight at room temperature. Upon returning, the mixture was transferred to a 125 mL Erlenmeyer flask with EA (25 mL), followed by the addition of brine and stirred vigorously. The mixture was transferred to a separatory funnel, and the aqueous layer was extracted with EA (3×30 mL). The organic layer was washed with brine (5×15 mL), dried over MgSO₄, gravity filtered, and concentrated under reduced pressure. The crude residue was purified by silica gel flash column chromatography using a combiflash with 100% hexanes for 4 min before alternating to a 15-25% EA gradient and then to a 25-47.2% EA gradient. The desired fraction tubes were collected and the solvent was evaporated under reduced pressure to yield NPET 1 as yellow solid (0.1 g, 0.19 mmol, 58% yield). R_{f}=0.36 (Hex:EA 1:1). M.p.: 133.4-136.9° C. ¹H NMR (400 MHz, CD₃CN) δ 9.08 (s, 1H), 7.96-7.91 (m, 2H), 7.60 (dd, J=8.5, 1.8 Hz, 1H), 7.55 (dd, J=5.2, 1.1 Hz, 1H), 7.43 (dd, J=3.7, 1.1 Hz, 1H), 7.39-7.28 (m, 5H), 7.12 (dd, J=5.2, 3.6 Hz, 1H), 5.56 (s, 1H), 5.43 (q, J=6.5 Hz, 1H), 5.02 (s, 2H), 3.00 (q, J=6.6 Hz, 2H), 1.89 (s, 2H), 1.56 (d, J=6.5 Hz, 3H), 1.44 (q, J=7.4 Hz, 2H), 1.36-1.23 (m, 3H). ¹³C{¹H} NMR (100 MHz, CD₃CN) δ 171.17, 157.28, 148.82, 139.11, 138.49, 134.61, 131.99, 131.80, 130.49, 129.38, 129.33, 128.78, 128.75, 128.61, 125.72, 122.50, 118.26, 92.03, 87.49, 78.78, 66.62, 40.94, 32.83, 29.73, 22.99, 21.24; FTIR (ATR) (cm⁻¹): 3328 (w), 3223 (w), 3108 (w), 3076 (w), 2995 (w), 2944 (w), 2864 (w), 2200 (w), 1886 (w), 1795 (w), 1763 (w), 1691 (s), 1658 (s), 1626 (w), 1597 (w), 1578 (w), 1551 (w), 1514 (m), 1439 (w), 1329 (s), 1267 (s), 1219 (w), 1176 (w), 1136 (w), 1098 (w), 1056 (m), 1026 (w), 930 (w), 908 (w), 838 (m), 750 (m), 715 (m), 696 (m), 654 (m), 611 (w). HRMS (DART-MS) m/z: [M+H]⁺ Calcd for C₂₇H₂₇N₃O₆SH 522.1699; found 522.0833. UV-vis (acetonitrile, λₘₐₓ, ε, concentration, path length): 345 nm, 2.8×10⁴ M⁻¹ cm⁻¹, 2.49×10⁻⁶ M, 1 cm. Emission (acetonitrile, λₘₐₓ): 494 nm.

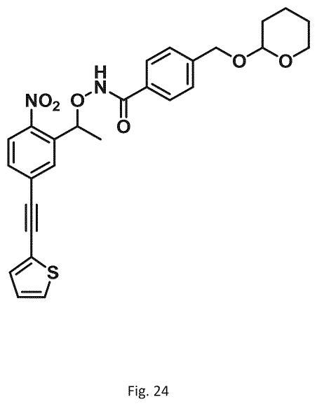

FIG. 24 shows a representation of 4-(hydroxymethyl)-N-(1-(2-nitro-5-(thiophen-2 ylethynyl) phenyl) ethoxy) benzamide (NPET 2).

Following the experimental of NPET 1, working in the dark as much as possible, compound 9 (0.10 g, 0.42 mmol, 1.2 equiv.) was placed into a 50 mL round bottom flask and dissolved in DMF (5 mL). PyBOP (0.23 g, 0.45 mmol, 1.3 equiv.) and 4-ethylmorpholine (0.26 mL, 2.08 mmol, 6.0 equiv.) were added while stirring for 20 min at room temperature, resulting in a color change from colorless to pink. Compound 7 (0.100 g, 0.35 mmol, 1.0 equiv.) was added to the reaction mixture, resulting in an orange color change. The reaction was allowed to stir at room temperature overnight. Upon returning, the mixture was transferred to a 125 mL Erlenmeyer flask with EA (25 mL), followed by the addition of brine (15 mL), and stirred vigorously. The mixture was transferred to a separatory funnel and the aqueous layer was extracted using EA (3×30 mL). The organic layer was washed using brine (5×15 mL), gravity filtered and concentrated under reduced pressure. The crude residue was purified by silica gel flash column chromatography using a combi flash with 100% hexanes to remove impurities before alternating to a 0-20% EA gradient. The desired fraction tubes were collected and the solvent was evaporated under reduced pressure to yield NPET 2 as an orange colored solid (0.15 g, 0.30 mmol, 87% yield). R_{f}=0.68 (Hex:EA 1:1). mp 58-64° C. ¹H NMR (400 MHz, CD₃CN) δ 9.67 (s, 1H), 7.99 (d, J=1.9 Hz, 1H), 7.87 (d, J=8.5 Hz, 1H), 7.54 (dd, J=8.5, 1.7 Hz, 1H), 7.51-7.44 (m, 3H), 7.36 (dd, J=3.7, 1.2 Hz, 1H), 7.29 (d, J=7.9 Hz, 2H), 7.04 (dd, J=5.2, 3.6 Hz, 1H), 5.54 (q, J=6.4 Hz, 1H), 4.64 (d, J=12.7 Hz, 1H), 4.58 (t, J=3.4 Hz, 1H), 4.39 (d, J=12.7 Hz, 1H), 3.74 (ddd, J=11.4, 8.3, 3.2 Hz, 1H), 3.44-3.34 (m, 1H), 1.74-1.54 (m, 5H), 1.53-1.38 (m, 4H). ¹³C{¹H} NMR (100 MHz, CD₃CN) δ 166.10, 148.48, 143.57, 138.69, 134.24, 131.70, 131.67, 131.65, 130.12, 128.91, 128.42, 127.97, 127.70, 125.31, 122.15, 98.68, 91.72, 87.10, 78.59, 68.45, 62.33, 30.90, 25.79, 20.97, 19.77; FTIR (ATR) (cm⁻¹): 3201 (w), 2940 (w), 2868 (w), 2206 (w), 1649 (m), 1602 (m), 1578 (m), 1517 (s), 1452 (w), 1387 (w), 1343 (s), 1303 (w), 1283 (m), 1218 (m), 1200 (w), 1182 (w), 1154 (w), 1117 (m), 1065 (m), 1030 (s), 1015 (s), 974 (w), 901 (s), 869 (w), 834 (s), 815 (w), 760 (w), 729 (m), 702 (s), 647 (w), 625 (w). HRMS (DART-MS) m/z: [M+H]⁺ Calcd for C₂₇H₂₆N₂O₆SH 507.1590; found 507.0833. UV-vis (acetonitrile, λₘₐₓ, ε, concentration, path length): 345 nm, 2.3×10⁴ M⁻¹ cm⁻¹, 2.57×10⁻⁶ M, 1 cm. Emission (acetonitrile, λₘₐₓ): 458 nm.\

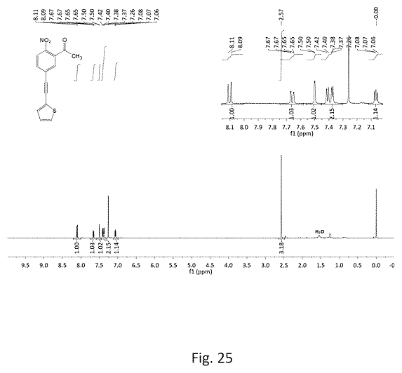

FIG. 25 shows characterization of ¹H NMR spectrum (400 MHz) of 4 in CDCl₃ at 25° C.

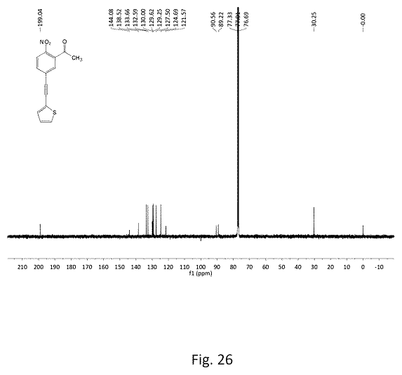

FIG. 26 shows characterization of ¹³C {¹H} NMR (100 MHz) of 4 in CDCl₃ at 25° C.

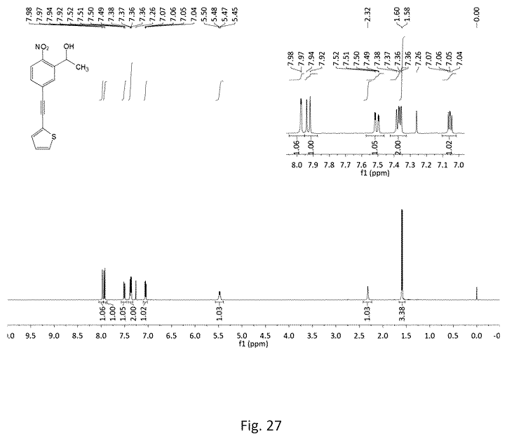

FIG. 27 shows characterization of ¹H NMR spectrum (400 MHz) of 5 in CDCl₃ at 25° C.

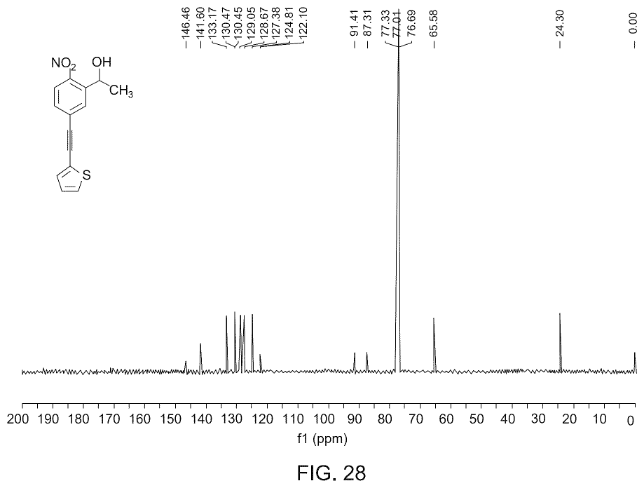

FIG. 28 shows characterization of ¹³C {¹H} NMR (100 MHz) of 5 in CDCl₃ at 25° C.

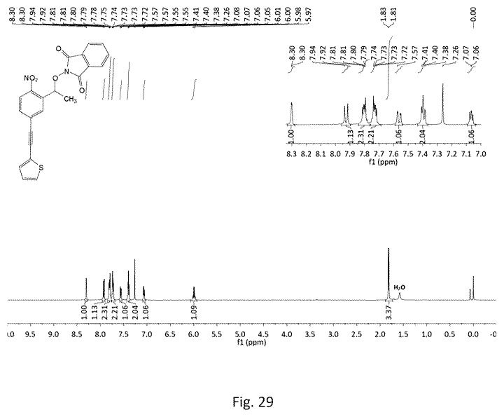

FIG. 29 shows characterization of ¹H NMR spectrum (400 MHz) of 6 in CDCl₃ at 25° C.

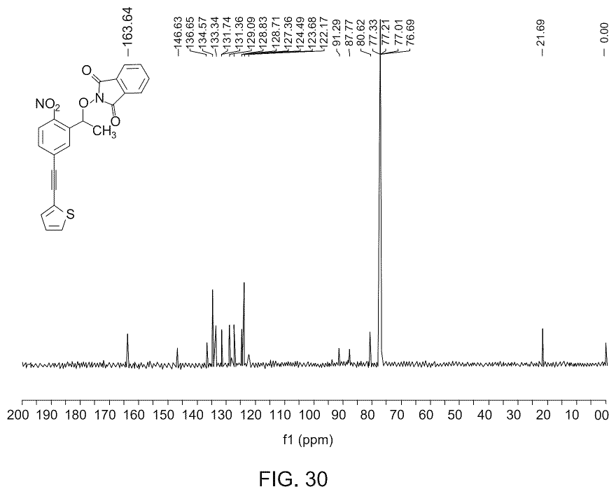

FIG. 30 shows characterization of ¹³C {¹H} NMR (100 MHz) of 6 in CDCl₃ at 25° C.

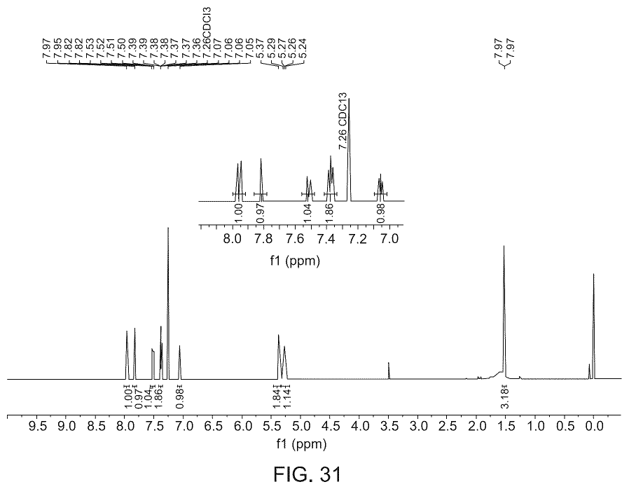

FIG. 31 shows characterization of ¹H NMR spectrum (400 MHz) of 7 in CDCl₃ at 25° C.

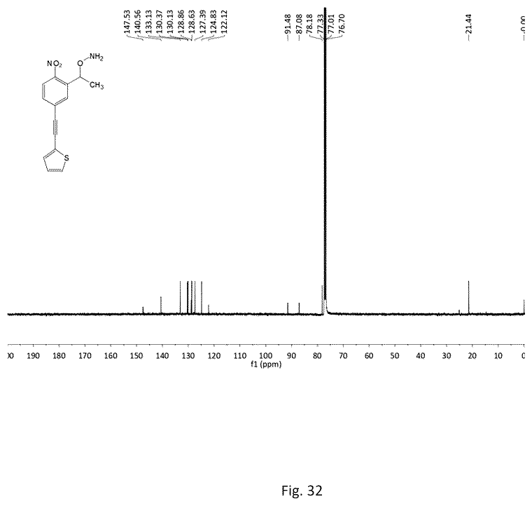

FIG. 32 shows characterization of ¹³C {¹H} NMR (100 MHz) of 7 in CDCl₃ at 25° C.

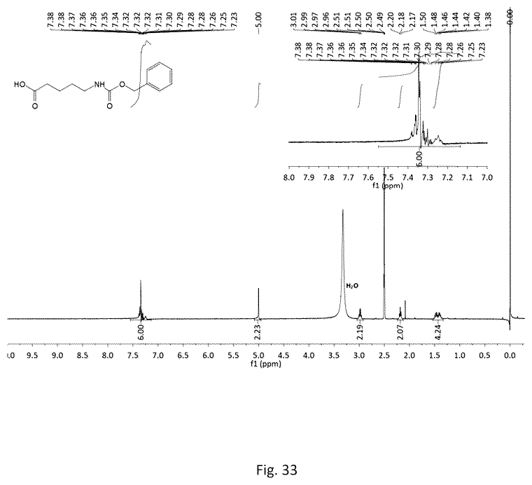

FIG. 33 shows characterization of ¹H NMR spectrum (400 MHz) of 8 in DMSO-d₆ at 25° C.

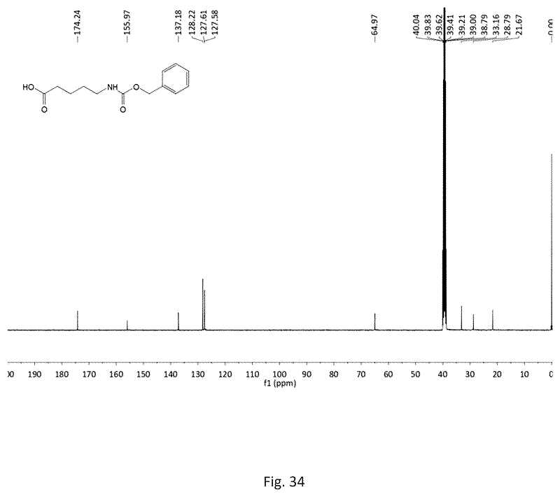

FIG. 34 shows characterization of ¹³C {¹H} NMR (100 MHz) of 8 in DMSO-d₆ at 25° C.

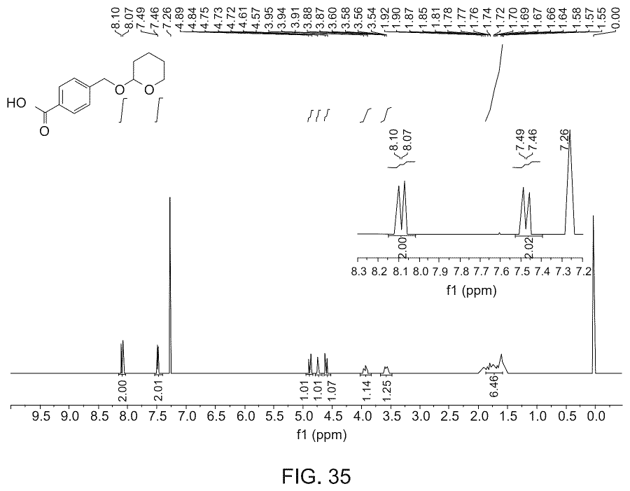

FIG. 35 shows characterization of ¹H NMR spectrum (300 MHz) of 9 in CDCl₃ at 25° C.

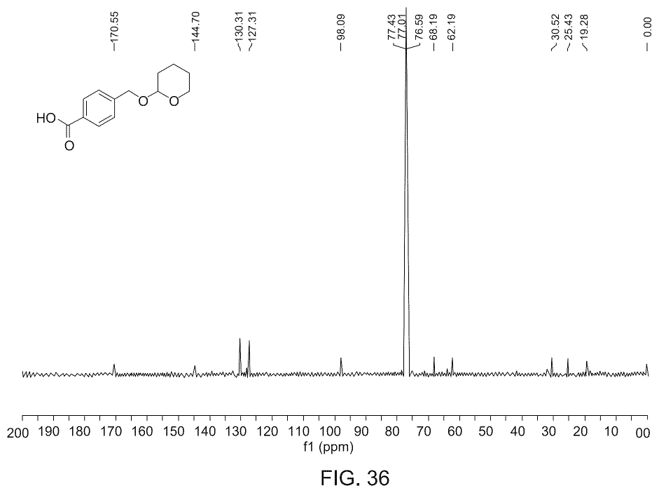

FIG. 36 shows characterization of ¹³C {¹H} NMR (75 MHz) of 9 in CDCl₃ at 25° C.

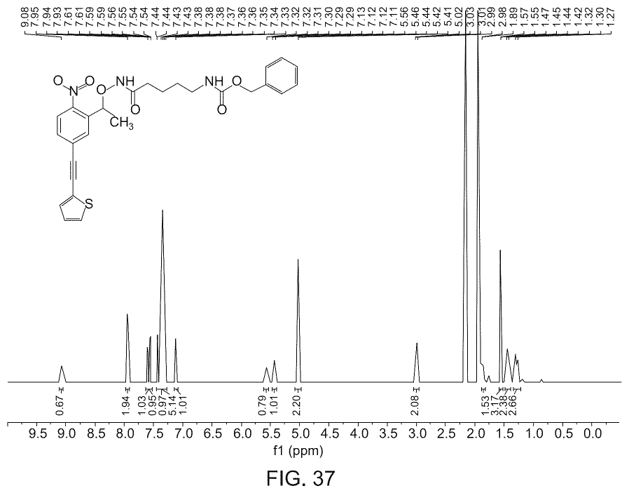

FIG. 37 shows characterization of ¹H NMR spectrum (400 MHz) of NPET 1 in CD₃CN at 25° C.

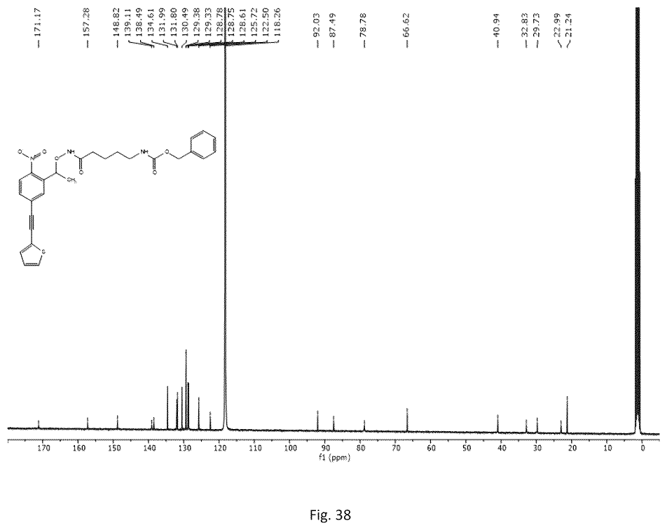

FIG. 38 shows characterization of ¹³C {¹H} NMR (100 MHz) of NPET 1 in CD₃CN at 25° C.

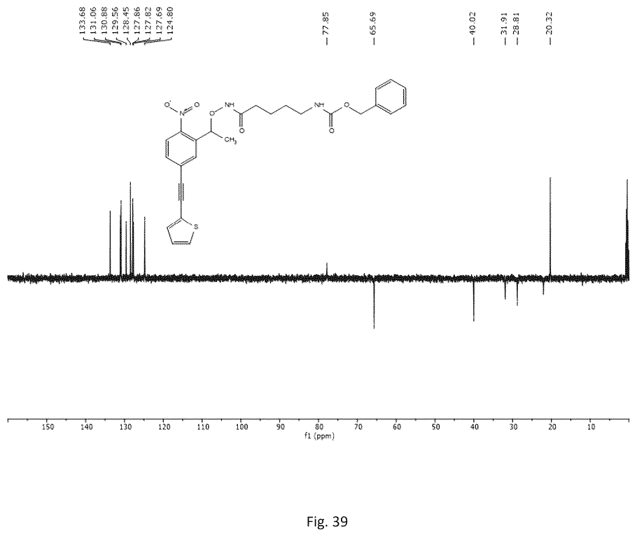

FIG. 39 shows characterization of ¹³C {¹H} DEPT 135 NMR (100 MHz) of NPET 1 in CD₃CN at 25° C.

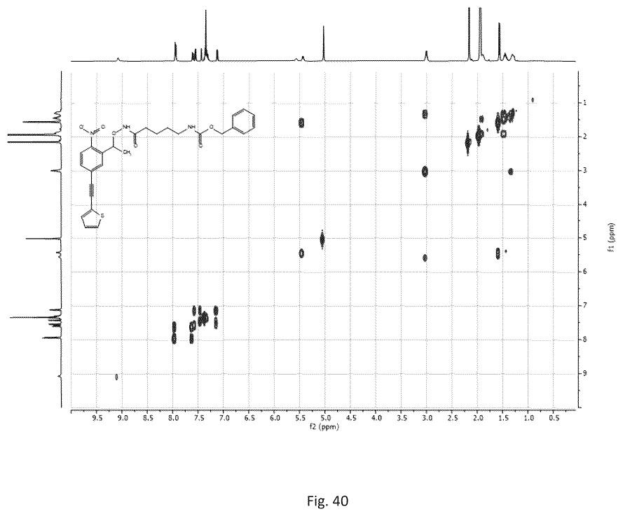

FIG. 40 shows characterization of ¹H-¹H COSY spectra of NPET 1 in CD₃CN (400 MHz).

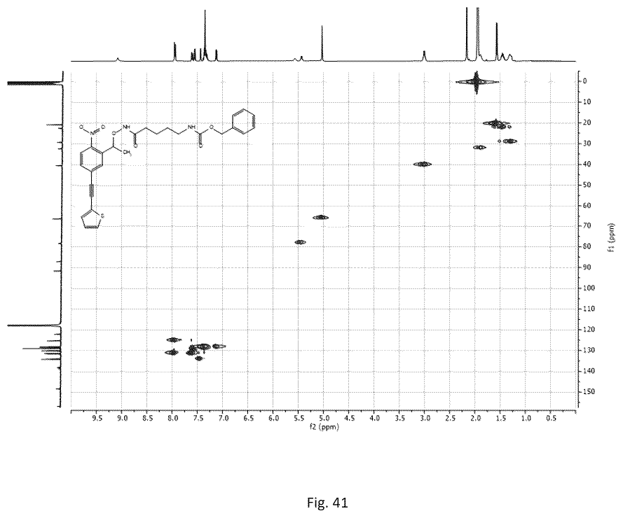

FIG. 41 shows characterization of HSQC NMR spectra of NPET 1 in CD₃CN (400 MHz).

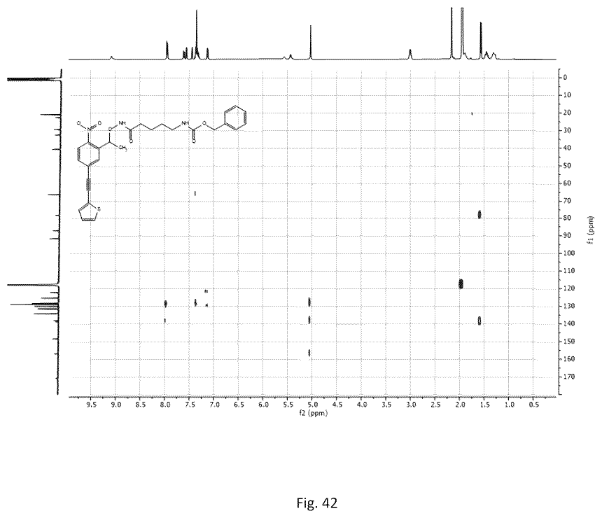

FIG. 42 shows characterization of HMBC NMR spectra of NPET 1 in CD₃CN (400 MHz).

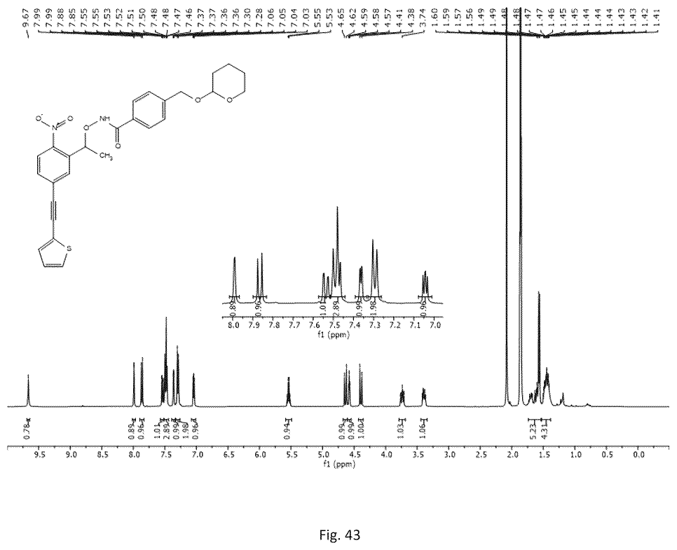

FIG. 43 shows characterization of ¹H NMR spectrum (400 MHz) of NPET 2 in CD₃CN at 25° C.

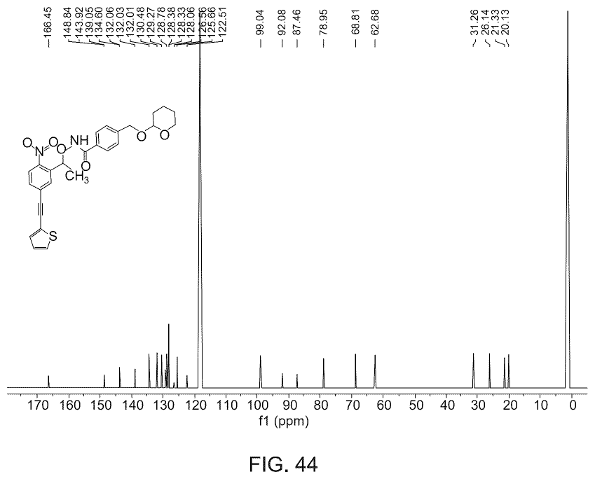

FIG. 44 shows characterization of ¹³C {¹H} NMR (100 MHz) of NPET 2 in CD₃CN at 25° C.

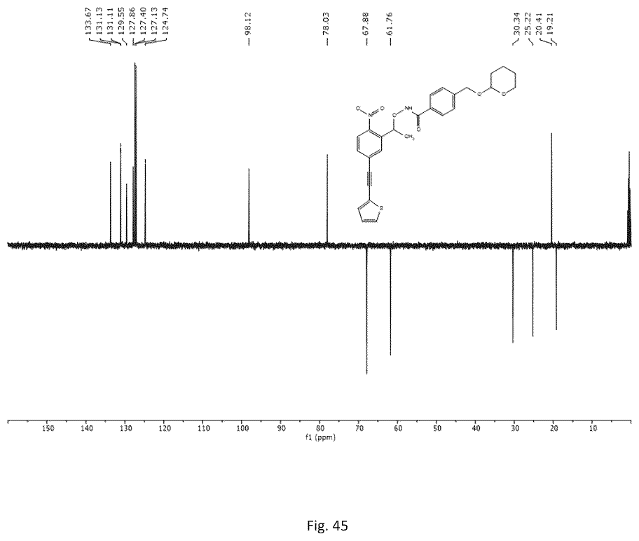

FIG. 45 shows characterization of ¹³C {¹H} DEPT 135 NMR (100 MHz) of NPET 2 in CD₃CN at 25° C.

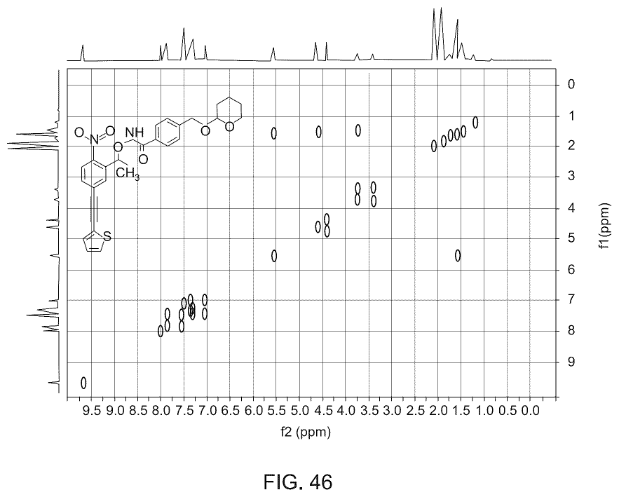

FIG. 46 shows characterization of ¹H-¹H COSY spectra of NPET 2 in CD₃CN (400 MHz).

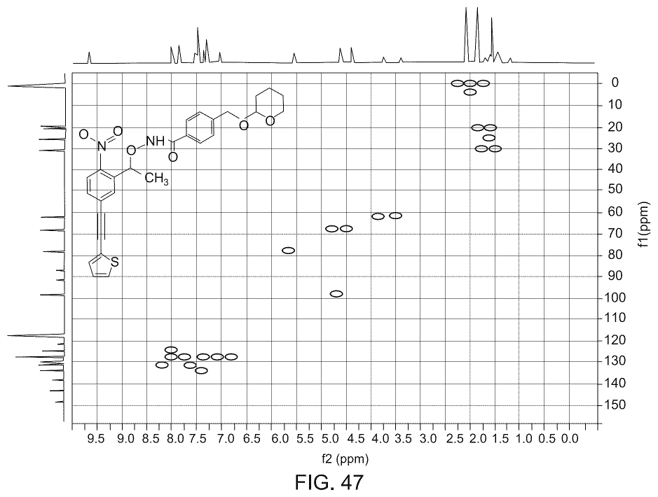

FIG. 47 shows characterization of HSQC NMR spectra of NPET 2 in CD₃CN (400 MHz).

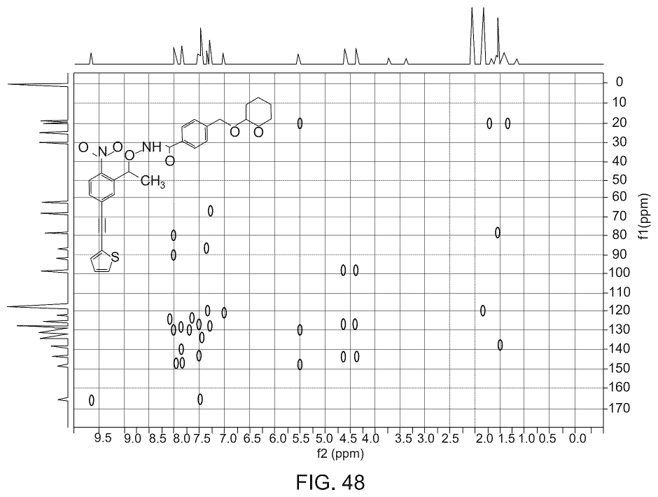

FIG. 48 shows characterization of HMBC NMR spectra of NPET 2 in CD₃CN (400 MHz).

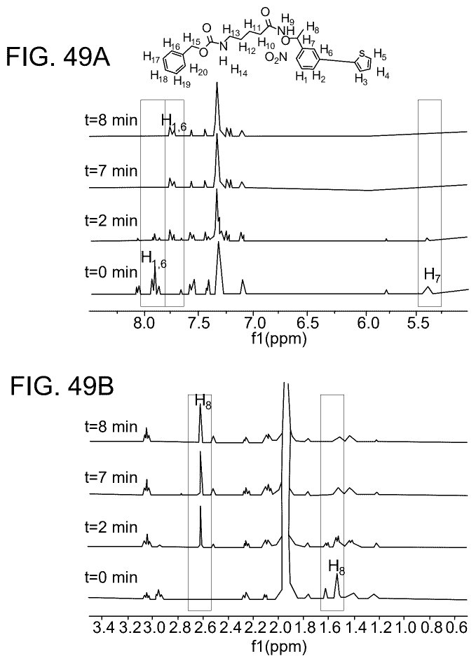

FIG. 49 shows ¹H NMR overlay photolysis of NPET 1 in CD₃CN:1M HCl (4:1 v/v) before (t=0 min) and after irradiation (t=8 mins) with a 365 nm UV lamp (30-watt 4-core LED). FIG. 49(A) shows ¹H-NMR cleavage trace from 8.5 ppm-5.00 ppm and FIG. 49(B) shows ¹H-NMR cleavage trace from 3.00 ppm-0.5 ppm. NPET 1 concentration=4.2 mM.

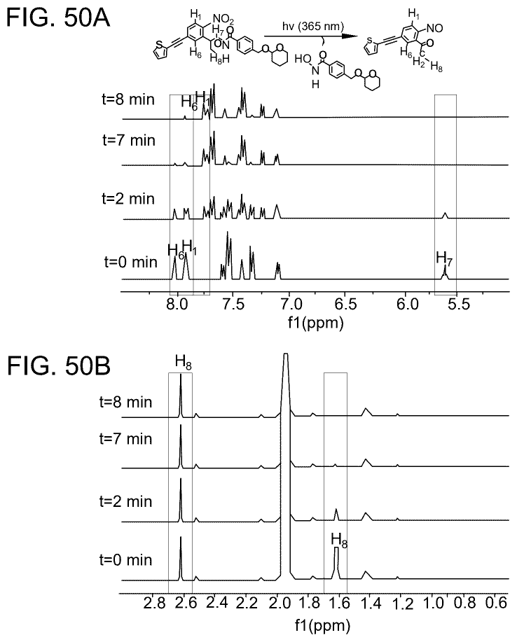

FIG. 50 shows ¹H NMR overlay photolysis of NPET 2 in CD₃CN:1M HCl (4:1) before (t=0 min) and after irradiation (t=8 mins) with a 365 nm UV lamp (30-watt 4-core LED). FIG. 50(A) shows ¹H-NMR cleavage trace from 8.5 ppm-5.00 ppm and FIG. 50(B) ¹H-NMR cleavage trace from 3.00 ppm-0.5 ppm. NPET 2 concentration=5.14 mM.

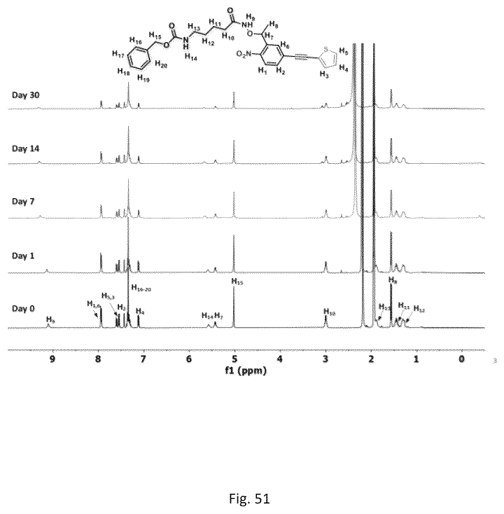

FIG. 51 shows ¹H NMR spectral overlay (400 MHz) of NPET 1 stability test over 30 days at ambient conditions.

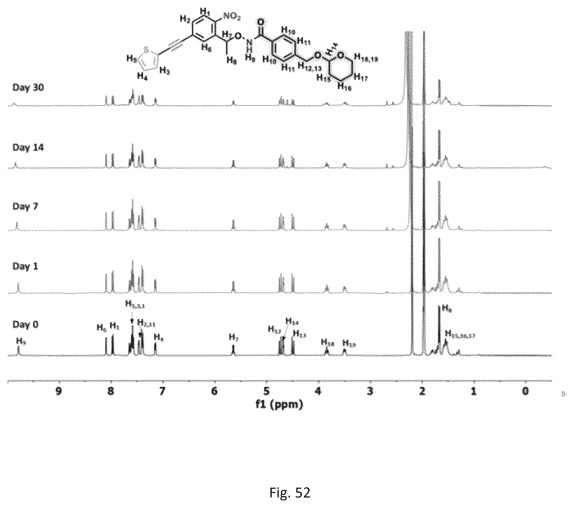

FIG. 52 shows ¹H NMR spectral overlay (400 MHz) of NPET 2 stability test over 30 days at ambient conditions.

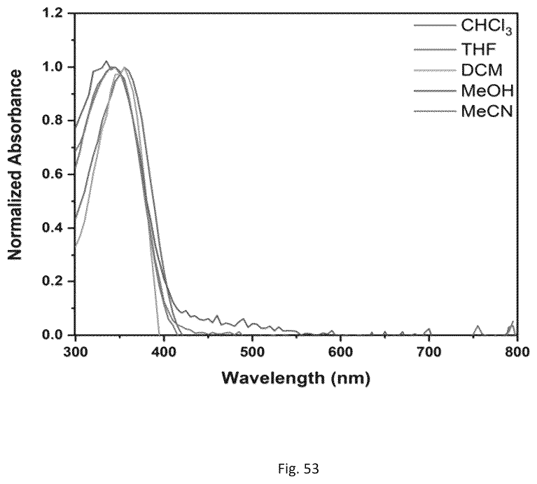

FIG. 53 shows normalized ultraviolet visible (UV-vis) spectrum of NPET 1 in different solvents of varying polarity (concentration=10⁻⁶ M).

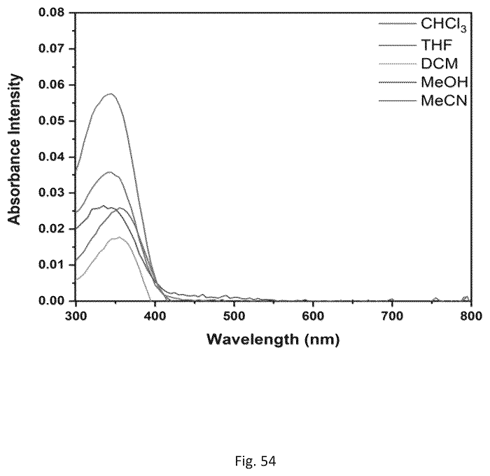

FIG. 54 shows UV-vis spectrum of NPET 1 in different solvents of varying polarity (concentration=10⁻⁶ M).

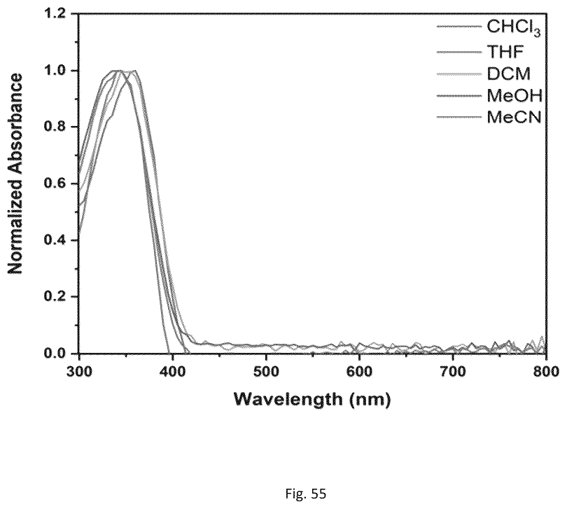

FIG. 55 shows normalized UV-vis spectrum of NPET 2 in different solvents of varying polarity (concentration=10⁻⁶ M).

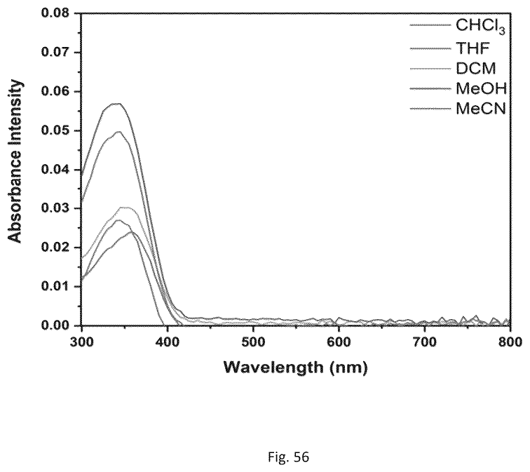

FIG. 56 shows UV-vis spectrum of NPET 2 in different solvents of varying polarity (concentration=10⁻⁶ M).

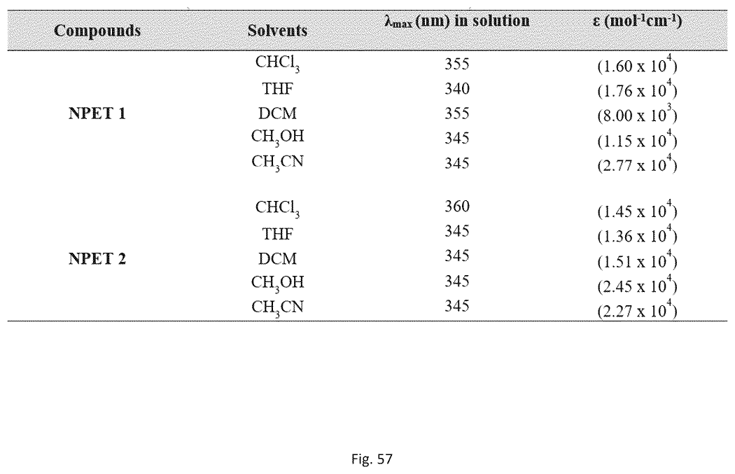

FIG. 57 is a table showing experimental UV-visible absorbance (λₘₐₓ) and molar absorption coefficient (ε) for NPET 1 and 2 in various solvents. (NPET concentration=10⁻⁶ M in solution).

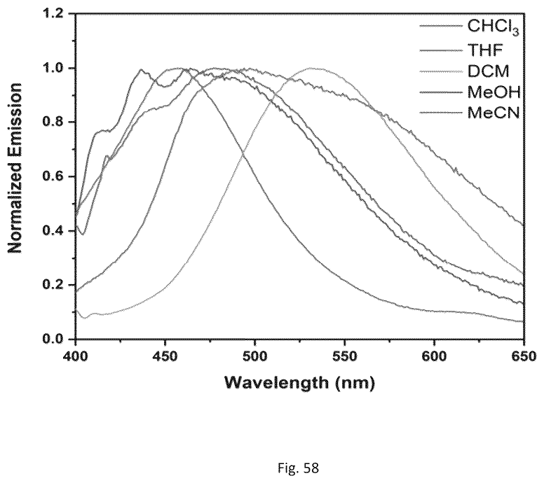

FIG. 58 shows normalized emission spectrum of NPET 1 in different solvents of varying polarity (concentration=10⁻⁵ M).

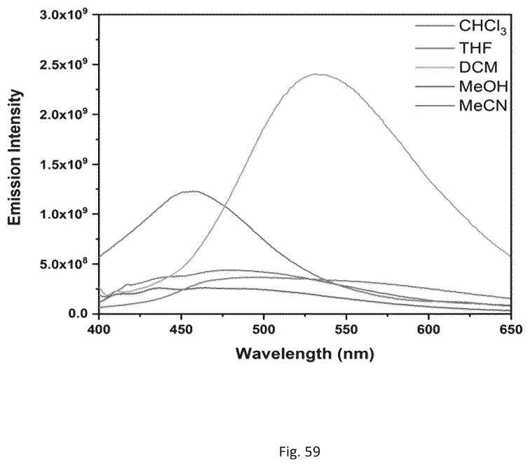

FIG. 59 shows emission spectrum of NPET 1 in different solvents of varying polarity (concentration=10⁻⁵ M).

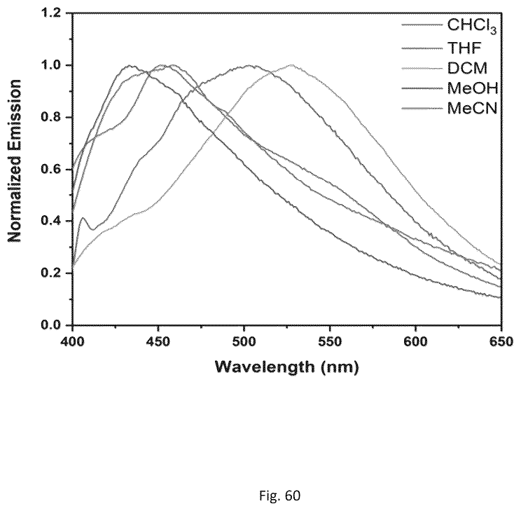

FIG. 60 shows normalized emission spectrum of NPET 2 in different solvents of varying polarity (concentration=10⁻⁵ M).

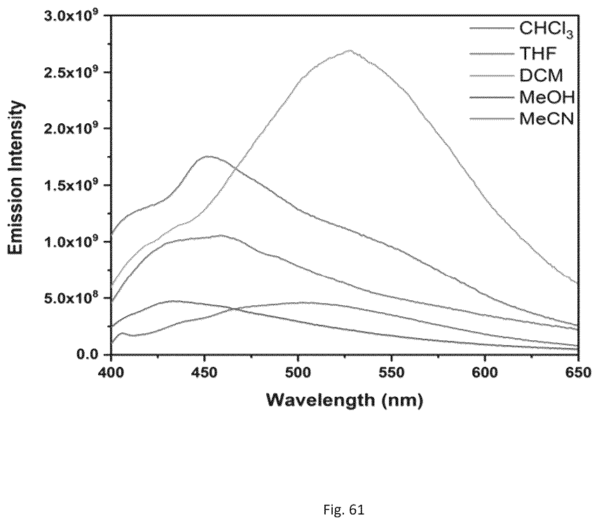

FIG. 61 shows emission spectrum of NPET 2 in different solvents of varying polarity (concentration=10⁻⁵ M).

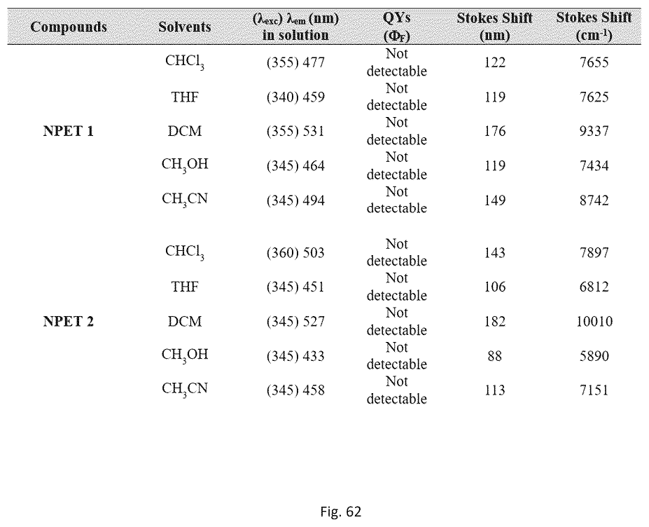

FIG. 62 is a table showing experimental emission (λₑₘ), QYs (Φ_{F}), and Stokes shift (nm) and (cm⁻¹) data for NPET 1 and 2 in various solvents. (NPET concentration=10⁻⁵ M in solution).

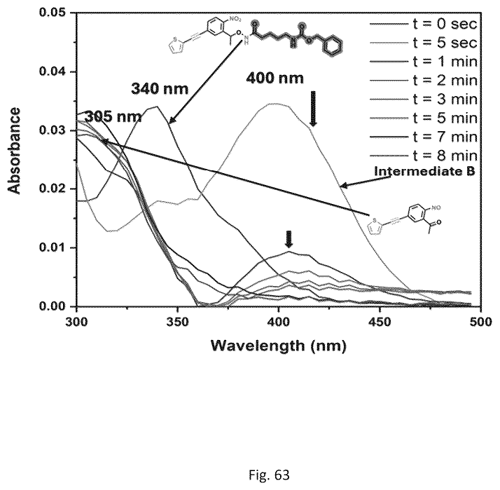

FIG. 63 shows a graph showing photolysis emission (λₑₘ), QYs (Φ_{F}), and Stokes shift (nm) and (cm⁻¹) data for NPET 1 and 2 in various solvents. (NPET concentration=10⁻⁵ M in solution).

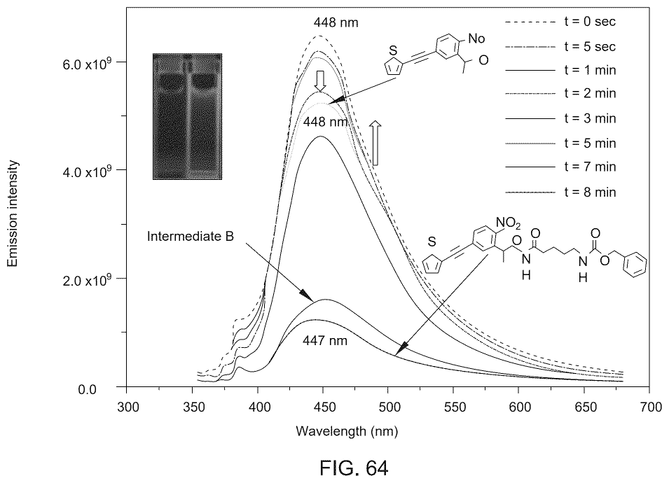

FIG. 64 shows a graph showing photolysis emission spectra of NPET 1.

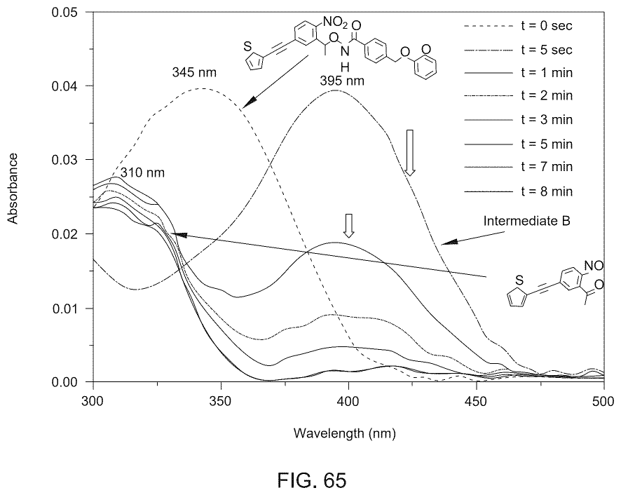

FIG. 65 shows a graph showing photolysis absorbance of NPET 2.

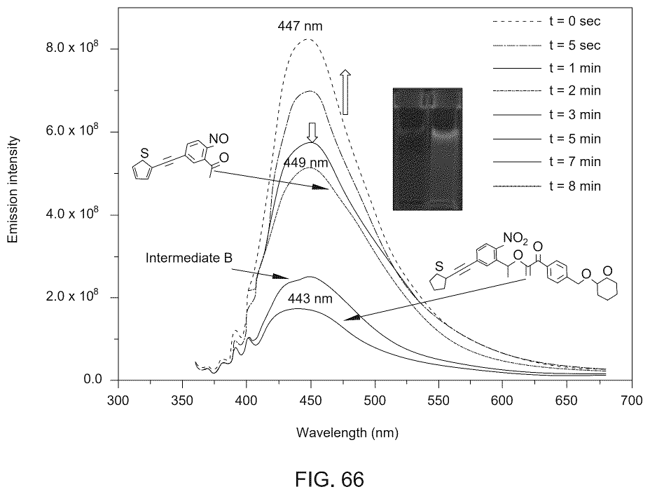

FIG. 66 shows a graph showing photolysis emission spectra of NPET 2.

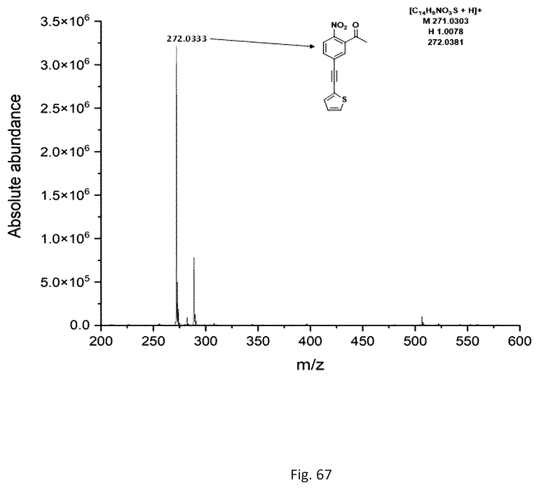

FIG. 67 shows a graph showing mass spectrum of compound 4.

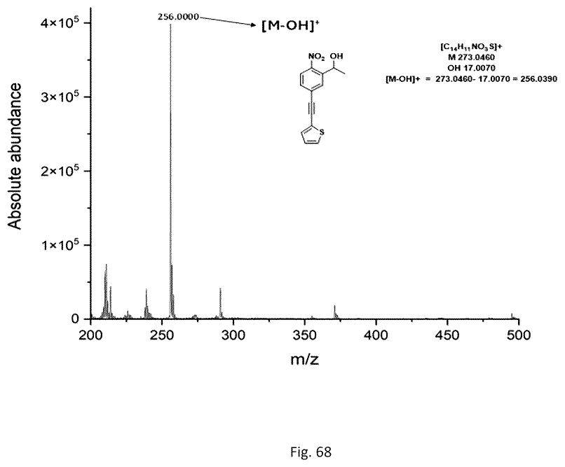

FIG. 68 shows a graph showing mass spectrum of compound 5.

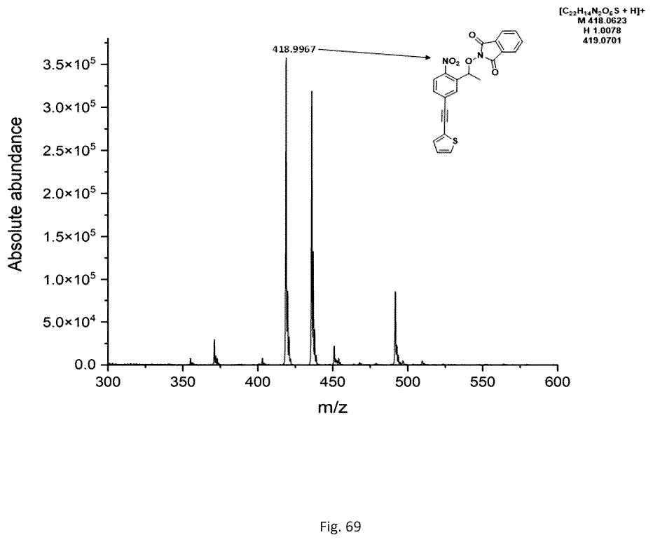

FIG. 69 shows a graph showing mass spectrum of compound 6.

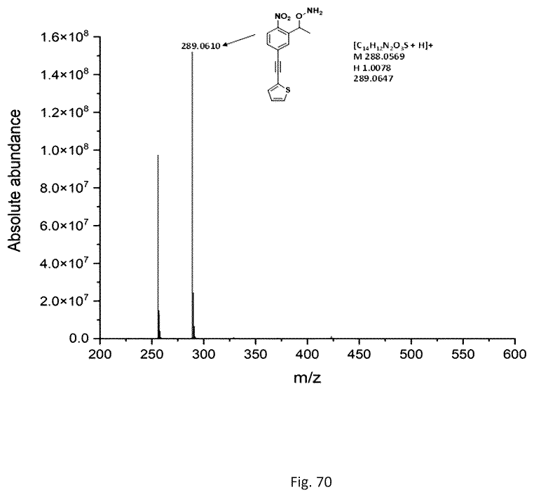

FIG. 70 shows a graph showing mass spectrum of compound 7.

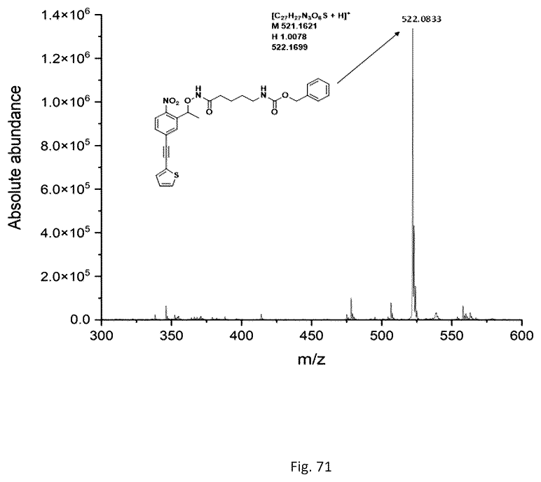

FIG. 71 shows a graph showing mass spectrum of NPET 1.

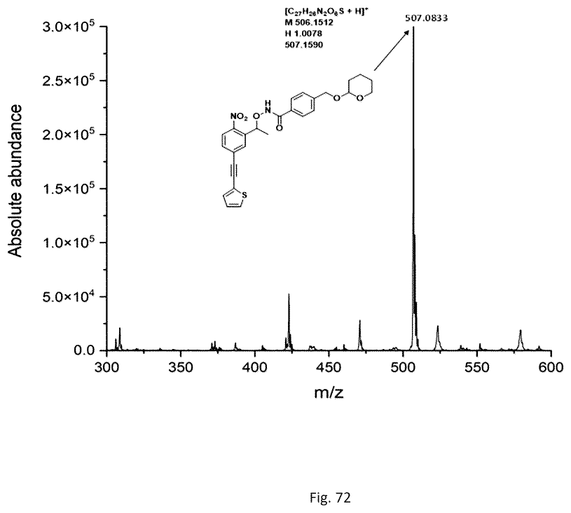

FIG. 72 shows a graph showing mass spectrum of NPET 2.

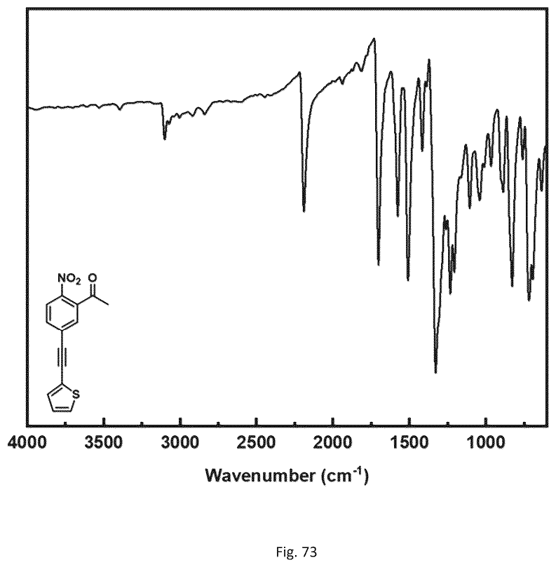

FIG. 73 shows a graph showing Fourier Transform Infrared Spectroscopy (FTIR) spectrum of 4.

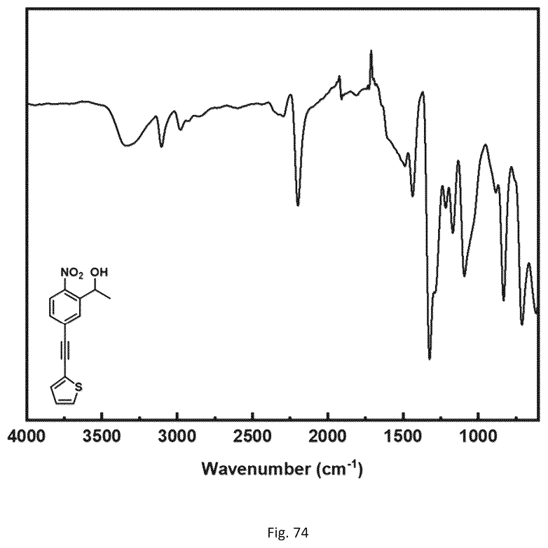

FIG. 74 shows a graph showing FTIR spectrum of 5.

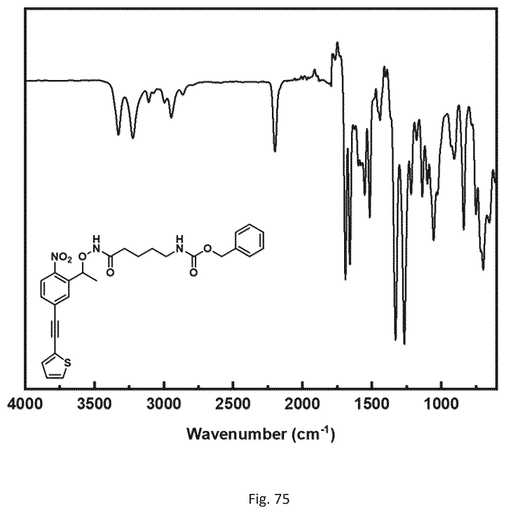

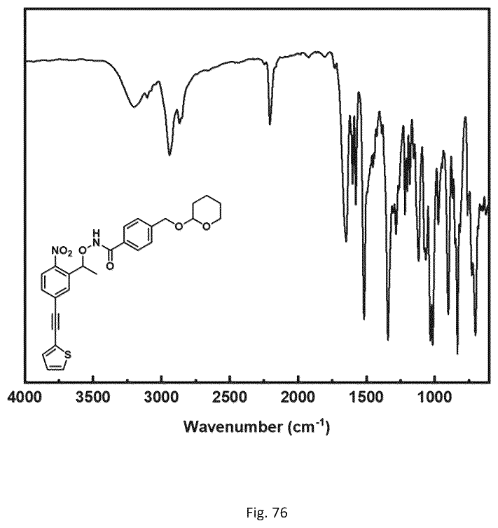

FIG. 75 shows a graph showing FTIR spectrum of NPET PPG 1. FIG. 76 shows a graph showing FTIR spectrum of NPET PPG 2. FT-IR spectra may be obtained from ¹H-NMR samples for NPETs 1 and 2 before and after irradiation in CD₃CN; 1M HCl (4:1) to validate the presence of the key functional groups of the HA derivatives and the nitrosoketone by-product (FIGS. 74 and 75). It is evident that the HAs from PPGs 1 and 2 were entirely cleaved, as the IR bands at 1516 cm⁻¹ and 1343 cm⁻¹, which correspond to the vibration of the —NO₂ group, were absent in the FTIR spectra for the photorelease products.

A new IR band at 1523 cm⁻¹ appears for both cleaved products, indicating the presence of the nitroso (—N═O) group on the nitrosoketone by-product. Additionally, a broad absorption band at 3337 cm⁻¹ corresponds to the O—H stretch of benzyl (5-(hydroxyamino)-5-oxopentyl)carbamate for cleaved PPG 1, while a broad absorption band at 3467 cm⁻¹ corresponds to the O—H stretch of N-hydroxy-4-(((tetrahydro-2Hpyran-2-yl)oxy)methyl)benzamide for cleaved PPG 2. These findings demonstrate the successfully uncaging of the HA derivatives from thiophene-based o-NB PPGs.

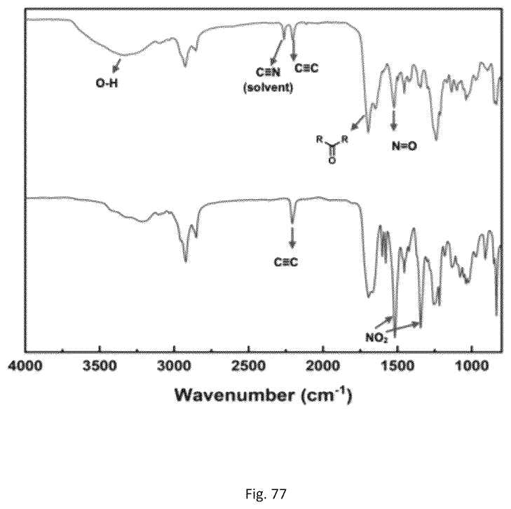

FIG. 77 shows a graph showing FT-IR spectral overlay of the uncleaved NPET 1 (bottom) and the photorelease products in CD₃CN (top) after irradiation with a 365 nm UV lamp (30-watt 4-core LED).

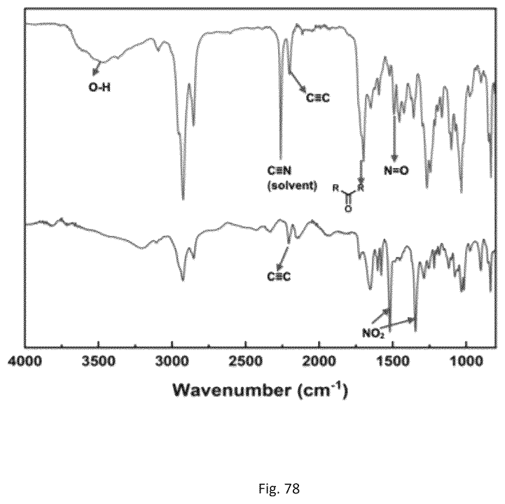

FIG. 78 shows a graph showing FT-IR spectral overlay of the uncleaved NPET 2 (bottom) and the photorelease products in CD₃CN (top) after irradiation with a 365 nm UV lamp (30-watt 4-core LED).

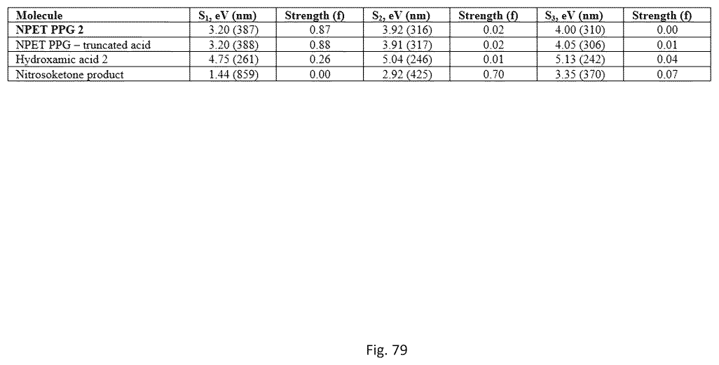

wB97X-D functional and cc-pVDZ basis set may be used. The wB97X-D hybrid functional includes a long-range correction, relevant to computing transitions involving charge transfer character, which are expected to appear in the nitrosoketone due to its push-pull architecture. As a reference, calculations may be performed using a hybrid functional that lacks the long-range correction, PBE0 (see FIG. 79). In general, PBEO gave more red-shifted energies due to the charge transfer nature of many of the low-lying states. All geometry optimizations and random phase approximation (RPA) TD-DFT calculations were carried out in acetonitrile using the C-PCM solvation model (i.e. setting the solvent dielectric 37.5 to represent acetonitrile).

FIG. 79 shows a table showing Time-Dependent Density Functional Theory (TD-DFT) computed vertical excitation energies (VEEs) for the first three singlet excited states of reactants and products computed with PBE0/cc-pVDZ in acetonitrile PCM. Excitation energies are reported in units of e V and nm (the latter in parentheses). The associated oscillator strength for each excitation is also shown.

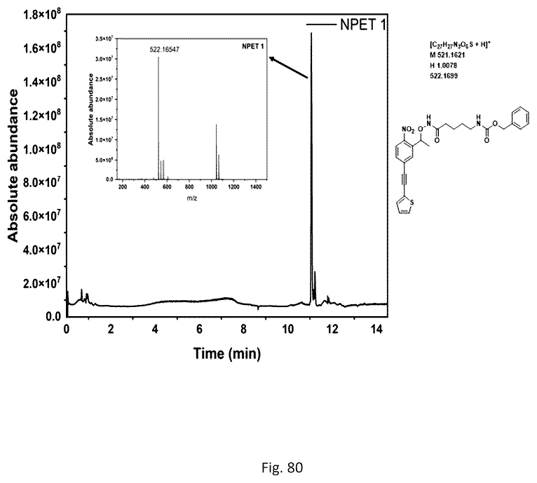

FIG. 80 shows a graph showing chromatograph and mass spectrum NPET 1 obtained by Liquid Chromatography-Mass Spectrometry (LCMS).

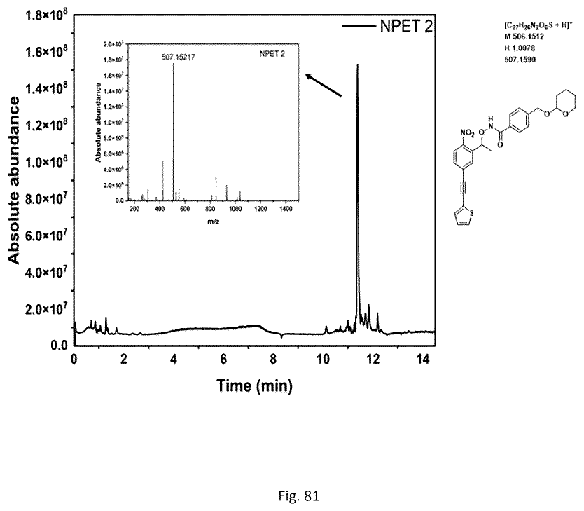

FIG. 81 shows a graph showing chromatograph and mass spectrum NPET 2 obtained by LCMS.

### Pro-Fluorescent Ethynylthiophene-Based o-Nitrobenzyl Photolabile Protecting Group for Hydroxamic Acid Synthesis

Photolabile protecting groups (PPGs) that enable real-time monitoring of uncaging processes are highly sought after for tracking product release during and after photolysis. Few PPGs facilitate direct detection of uncaging events through a fluorescence signal, with o-nitrobenzyl (o-NB) PPG derivatives being the only known examples exhibiting pro-fluorescent properties. The present disclosure broadens the scope of accessible pro-fluorescent o-NB PPGs for direct monitoring of product release by reporting two new pro-fluorescent, ethynylthiophene-based, and visible light-absorbing o-NB PPGs, referred to as NPETs 1 and 2. UV-Vis spectroscopy confirmed the complete cleavage of hydroxamic acid (HA) derivatives from NPETs 1 and 2, as evidenced by a blue shift and reduced absorbance intensity. This step likely proceeds through an aci-nitro intermediate, supported by both spectroscopic and computational examinations. The present disclosure assesses the released HA products by monitoring the corresponding increase in fluorescence intensity, which corresponds to the co-generated nitrosoketone by-product. The 4-fold and 3-fold increase in fluorescent intensity for NPETs 1 and 2, respectively, was easily observable with the naked eye. Time-course ¹H-NMR experiments revealed that NPET 2 exhibits greater stability than NPET 1, showing only minor degradation after 30 days at ambient conditions. (TD)-DFT calculations revealed that the nitrosoketone by-product emission occurs from the S₂ singlet excited state, violating Kasha's rule. This study highlights the efficacy of pro-fluorescent, ethynylthiophene-based o-NB PPGs in facilitating precise photoreactions under mild acidic conditions. Their pro-fluorescence response and minimal degradation under ambient conditions indicate their potential for application in releasing synthetically difficult-to-synthesize functional groups.

### Introduction

In chemistry and materials research, selectively protecting and deprotecting functional groups are critical, especially for lengthy synthetic routes where the number of functional groups requiring protecting groups (PGs) typically increases. Conventional methods such as strongly acidic, strongly basic, reductive, and oxidative conditions, are generally used to remove traditional PGs, which can potentially degrade the protected molecule. However, this drawback is circumvented by the alternative use of photolabile protecting groups (PPGs), which allow selective removal of PGs using light as the activator. This light-triggered functionality enables selective, spatially controlled release of molecules of interest under mild conditions. Such light-triggered functionalities have paved the way for transformative advances, ranging from fine-tuned drug delivery systems to the controlled construction of biomolecular architectures.

The first example of PPGs, the ortho-nitrobenzyl (o-NB) group containing a C—H bond at the ortho-position, was developed in 1966 by Barltrop et al. as a more desirable approach to addressing the issue of potential degradation of the protected molecule during the deprotection of PGs using conventional methods. This approach was later improved by incorporating a methyl (—CH₃) group at the benzylic position orthogonal to the —NO₂ group in response to the formation of an unwanted nitrobenzaldehyde by-product during PPG deprotection. This detrimental by-product underwent a side reaction with amine-release products, resulting in low yields of released products. Since Barltrop's discovery, chemists have been working on developing new pro-fluorescent PPGs and increasing their uncaging efficiency. Formation of a fluorescent by-product is advantageous because it provides a direct method for monitoring and quantifying the released products during and after photolysis.

There are currently limited examples of PPGs, such as cinnamate-based or thiochromone-based molecules, that enable the detection of uncaging events through the appearance of a fluorescence signal. Similarly, only one series of o-NB PPG with pro-fluorescent properties is known from the literature: the 1-(2-nitrophenyl)-2-phenylethan-1-ol PPG derivatives developed by Specht et al. Therefore, it is important to expand the range of available o-NB PPGs to include those with pro-fluorescent properties capable of directly monitoring and quantifying the released products of photo-cleavage. The present disclosure reports the first and sole example of a pro-fluorescent, ethynylthiophene-based o-NB PPG that absorbs light in the near-visible region of the electromagnetic spectrum for the synthesis of hydroxamic acids (HAs) in high yields. It also co-generates a readily detectable fluorescent by-product that can be used to indicate the formation of HA products. HAs, a class of organic compounds, have garnered extensive attention due to their diverse applications as metal chelators for the removal of toxic metals from seawater, precursors of several anti-cancer drugs, dyes, optoelectronic devices, and polymer architectures. However, the synthesis of HAs is widely recognized as challenging, and their purification can be difficult because HAS are highly reactive and often form a mixture of poly-substituted by-products under conventional reaction conditions. The present disclosure presents the design, synthesis, and application of two new ethynylthiophene-based o-NB PPGs, referred to as NPETs 1 and 2, with a nitrosoketone pro-fluorescent by-product as a more promising approach to addressing the issues associated with HA synthesis and their purification. These findings will contribute to the development of future ethynylthiophene-based o-NB PPGs with pro-fluorescent uncaging properties, capable of photo-releasing other difficult-to-synthesize functional groups such as carboxylic acids bonded to poor leaving groups like alcohols, phenols, and thiols, wherein the initially photo-cleaved carbonic or thiocarbonic acid would be unstable and undergo decarboxylation, resulting in the more readily obtainable free alcohols or thiols.

### Results and Discussion

### Design

Designing the 2(2-(4-nitrophenyl)ethynyl)thiophene (NPET) PPG to absorb near the visible region of the electromagnetic spectrum involved three important considerations: (1) the core of the PPG should possess a coplanar geometry and be composed of an extended conjugated z-system; (2) a push-pull character needs to exist throughout the PPG by incorporating electron-donating and/or-withdrawing groups; and (3) the conformation of the PPG should be rigid by regulating a linker between the electron-donating and-withdrawing groups, hindering free rotation throughout the PPG scaffold. This last requirement can be achieved by incorporating alkyne or alkene moieties as linkers.

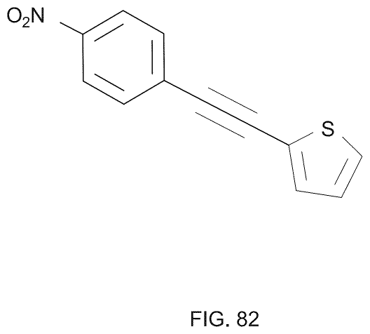

The planarity of the PPGs can significantly impact the flow and overall distribution of z-electrons, influencing PPGs'performance. To achieve this desired geometry, the present disclosure incorporates an alkyne group (pink in FIG. 82) as a bridge between the thiophene and the o-NB moieties. Additionally, the present disclosure uses a —NO₂ group (red in FIG. 82) as an electron-withdrawing moiety at the para-position to the electron-rich thiophene (orange in FIG. 82) to create a push-pull effect throughout the conjugated π-system. This combination has been shown to lead to important optical properties, such as increased absorption and emission wavelengths. Incorporating a non-carbon atom (sulfur in our case) into the scaffold of the PPG not only enhanced its structural integrity but also lead to significance optical properties such as red-shifting both the maxima of absorption and emission. Thiophenes are particularly valuable for manipulating the electronic properties of various organic molecules, making them an ideal choice for controlling the absorbance and the photo reactivity of our novel PPGs.

FIG. 82 shows molecular structure of NPET PPG scaffold.

### Syntheses

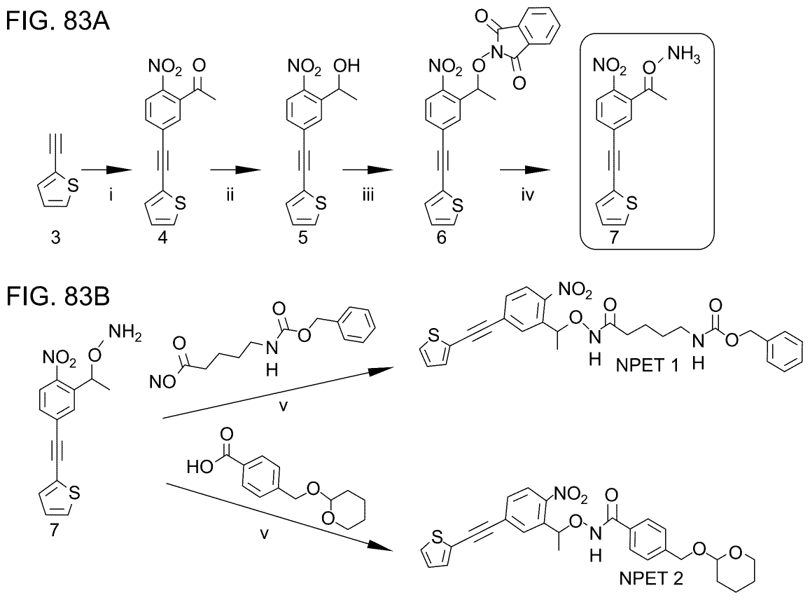

The NPET PPGs were synthesized in two steps. Initially, the NPET PPG scaffold, which contained a hydroxylamine moiety, was synthesized (FIG. 83A). Subsequently, this scaffold was reacted with two carboxylic acid derivatives to generate NPETs 1 and 2, respectively (FIG. 83(B)).

FIG. 83 shows a synthetic route to synthesize NPETs 1 and 2. FIG. 83(A) shows reagents and conditions to synthesize hydroxylamine 7: (i) 1-(5-bromo-2-nitrophenyl)ethan-1-one, PdCl₂(PPh₃)₂, CuI, Et₃N, N₂, (85° C., 2 h, 79%); (ii) NaBH₄, 5:1 THF:MeOH, (−78° C.-rt, 15 min, 79%); (iii) N-hydroxyphthalimide, PPh₃, DIAD, THF, (−5° C.-rt, 14 h, 94%); (iv) NH₂NH₂·H₂O, MeOH (rt, 4 h, 81%). FIG. 83(B) shows reagents and conditions to synthesize NPETs 1 and 2 from hydroxylamine 7: (v) 4-ethylmorpholine, PyBOP, DMF (rt, 24 h, 58% and 87%, respectively).

The multistep synthesis began with a simple Sonogashira coupling reaction between 3 and 1-(5-bromo-2-nitrophenyl)ethan-1-one, catalyzed by copper iodide (CuI), yielded 4 in good yields. This ketone was then reduced to alcohol 5, which was subsequently coupled to N-hydroxyphthalimide to produce 6. Finally, 6 was hydrolyzed with hydrazine hydrate, facilitating deprotection and the formation of hydroxylamine 7.

The hydroxylamine 7 served as the starting material for the synthesis of the two new NPETs 1 and 2, which are functionalized with an alkyl 8 and phenyl group 9 respectively. Notably, compound 7 can also be coupled with any number of carboxylic acid derivatives to generate any HA derivatives, highlighting the versatility of the hydroxylamine 7. The structures of previously unreported compounds and NPETs 1 and 2 were confirmed by ¹H, ¹³C{¹H} NMR spectroscopies, 2D NMRs (COSY, DEPT 135, HSQC, HMBC) for NPETs 1 and 2, high resolution mass spectrometry, LCMS for NPETs 1 and 2, and FT-IR (see ESI⁵⁵⁴). All compounds were stable under ambient conditions.

Photophysical and photochemical characterization After successfully synthesizing and fully characterizing NPETs 1 and 2, their photolytic release properties were investigated by irradiating both samples in a 4/1 (v/v) acetonitrile/1M HCl mixture, and the absorbance and emission wavelengths were observed to change over time using UV-Vis and emission spectroscopies. Acetonitrile (MeCN) was selected as the solvent for photolysis due to its relatively inertness under photolysis conditions. It is also well established in the literature that polar aprotic solvent like MeCN stabilize charged species and promote intramolecular charge transfer, leading to a red-shift in emission, which is particularly beneficial when photolysis involves charge separated intermediates like NPETs 1 and 2. To promote hydrolysis during photolysis, 1M HCl was added to the sample, as the HAs were not fully cleaving and the reaction was stalling at intermediate D.

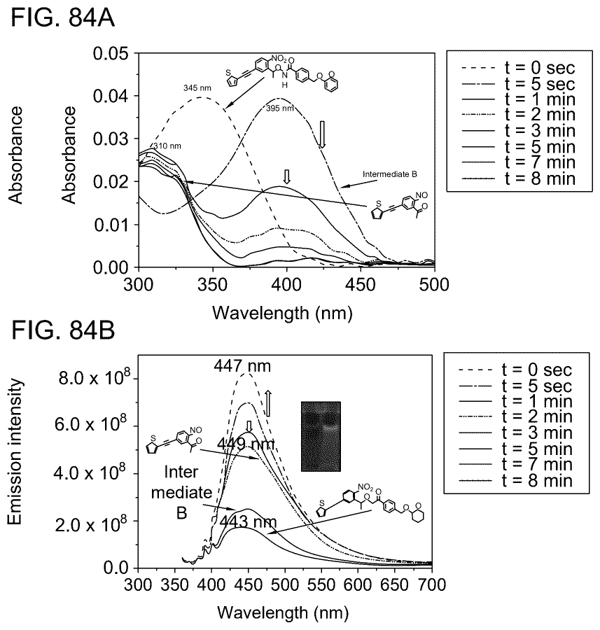

Before irradiation (at time=0 second), an absorbance scan was taken with a λₘₐₓ of 340 nm (ε=2.12×10⁴ M⁻¹ cm⁻¹) corresponding to the caged NPET 1 (FIGS. 63) and 345 nm (ε=2.38×10⁴ M⁻¹ cm⁻¹) corresponding to the caged NPET 2 (FIG. 84A), respectively. The samples were then irradiated with a 365 nm UV lamp (30 watt 4-core LED) at 5-seconds interval, followed by 1-minute intervals for 8 minutes while recording the λₘₐₓ and relevant absorbances.

After the initial 5 seconds, a new red-shifted absorbance band with a λₘₐₓ of 400 nm (ε=2.14×10⁴ M⁻¹ cm⁻¹) was observed for NPET 1 (FIG. 63) and 395 nm (ε=2.38×10⁴ M⁻¹ cm⁻¹) for NPET 2 (FIG. 84A), indicating the formation of the aci-nitro intermediate (intermediate B). It is well-established in the literature that the primary photoreaction of 2-nitrobenzyl compounds involves an intramolecular hydrogen atom-transfer, leading to the formation of aci-nitro tautomers, which are easily identified by their strong absorption near 400 nm. These tautomers are also known to represent the rate-limiting step in product release, as their decay is commonly used to estimate the release kinetics of the protected species.

Generation of this intermediate was monitored, with a gradual decrease in absorbance intensity until remaining constant after 8 minutes of constant irradiation, indicating complete cleavage of the N-hydroxy-4-(((tetrahydro-2H-pyran-2-yl)oxy)methyl)benzamide from the NPET 2 and benzyl (5-(hydroxyamino)-5-oxopentyl)carbamate from NPET 1 (FIG. 63). The resulting by-product is a nitrosoketone with a λₘₐₓ of 305 nm (ε=8.91×10³ M⁻¹ cm⁻¹) and 310 nm (ε=7.32×10³ M⁻¹ cm⁻¹) (FIGS. 63 and 83(A), respectively). The presence of an isosbestic point at 330 nm (the intermediate photoproduct and nitrosoketone by-product) indicates that a clean, degradation-free photo-cleavage occurred with stable released photoproducts.

FIG. 84(A) shows photolysis absorbance spectra for NPET 2 in 4:1 (v/v) MeCN:1M HCl after intervals of irradiation at 365 nm (concentration=10⁻⁶ M). FIG. 84(B) shows emission spectra for NPET 2 during and after irradiation at 365 nm showing an increase in fluorescence intensity (concentration=10⁻⁵ M).

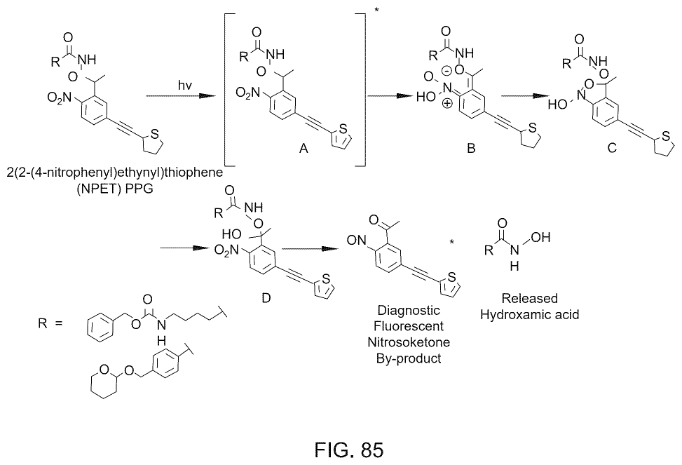

The proposed mechanism for the photolysis of NPET PPGs is provided in FIG. 85. The present disclosure hypothesizes that the photolysis mechanism of NPETs 1 and 2 is similar to that of o-NB deprotection, which has been extensively explored and reviewed. In brief, when exposed to irradiation, the NPET PPG molecule is excited from its ground state to an excited state (FIG. 85A). Subsequently, the —NO₂ group from this excited species abstracts a hydrogen intramolecularly, forming the aci-nitro intermediate (FIG. 85B). The aci-nitro intermediate then undergoes irreversible cyclization to produce the cyclic intermediate (FIG. 85C). Following ring-opening, the hemiacetal intermediate (FIG. 85D) is formed, releasing the nitrosoketone by-product and ultimately resulting in the HA products.

FIG. 85 shows proposed mechanism for the cleavage of NPET PPGs.

The present disclosure monitors the photo-induced changes in fluorescence emission changes for NPETs 1 and 2 in a 4/1 (v/v) MeCN/1M HCl mixture. The photolysis emission spectra for NPET 1 are shown in FIG. 64 of the supporting information. Prior to photolysis, both NPET PPGs displayed no fluorescence, with an emission λₘₐₓ at 447 and 443 nm for NPETs 1 and 2, respectively. These emissions were not detectable with the naked eye (see inset photographs in FIG. 83(B) and 64). Upon 5 seconds of irradiation, NPET 1 exhibits an increase in emission intensity which continued to rise and then plateaued after 3 minutes with an emission λₘₐₓ of 448 nm. At 5 minutes, the emission intensity began to decrease and remand stable through 8 minutes with an emission λₘₐₓ of 448 nm (QY=0.20%). This final emission band is attributed to the nitrosoketone by-product, showing a 4-fold increase in emission intensity. A similar trend was observed for NPET 2, which showed a 3-fold increase in emission after 8 minutes of irradiation, also with an emission λₘₐₓ of 449 nm (QY=1.30%) corresponding to the same nitrosoketone by-product (FIG. 84B). A side-by-side visual comparison of caged and uncaged NPET 2 is displayed in the inset photo in FIG. 84B, highlighting the fluorescent enhancement. Initially, both NPET PPGs are non-emissive to the naked eye, however, upon full photolysis, they yielded a distinctly fluorescent nitrosoketone by-product, demonstrating the pro-fluorescent quality of these PPGs (FIGS. 83(B) and 64).

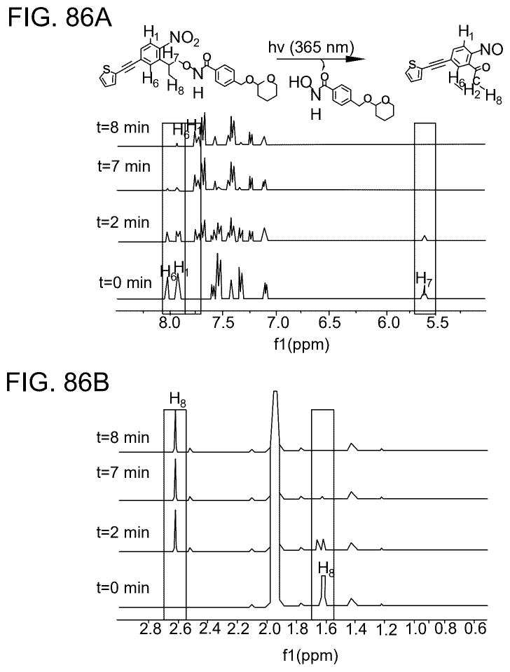

To investigate the formation of the HA derivatives and the nitrosoketone fluorescent by-product, ¹H NMR spectroscopy analysis was conducted on NPETs 1 and 2 (see FIG. 86 for NPET 2 and FIG. 49 for NPET 1). The structures of N-hydroxy-4-(((tetrahydro-2H-pyran-2-yl)oxy)methyl)benzamide and the nitrosoketone fluorescent by-product derived from NPET 2 were confirmed by acquiring ¹H NMR in CD₃CN:1M HCl (4:1) before and after 8 minutes of irradiation with a 365 nm UV lamp (30-watt 4-core LED).

Consistent with the UV-vis spectroscopy results, the HA from NPET 2 underwent complete cleavage after 8 minutes, as evidenced by the disappearance of the H₇ proton signal at 5.57 ppm, directly bonded to the benzylic carbon (red) in the position ortho to the nitro group. Before photolysis, this signal displayed a quartet splitting at 5.57 ppm since it is directly bonded to the methyl group. Following 8 minutes of photolysis, the signal at 5.57 ppm disappeared, confirming the formation of the ketone in the nitrosoketone by-product. Additionally, the H₈ proton from the methyl group, which originally resonated at 1.61 ppm, became deshielded and shifted from a doublet to a singlet at 2.62 ppm, further supporting the formation of the ketone in the nitrosoketone fluorescent by-product.

FIG. 86 shows ¹H NMR overlay photolysis of NPET PPG 2 in CD₃CN:1M HCl (4:1) before (t=0 min) and after irradiation (t=8 mins) with a 365 nm UV lamp (30-watt 4-core LED). FIG. 85(A) shows ¹H-NMR cleavage trace from 8.5 ppm-5.00 ppm and FIG. 86(B) shows ¹H-NMR cleavage trace from 3.00 ppm-0.5 ppm. (NPET 2 concentration=5.14 mM).

A similar trend was observed for the ¹H-NMR analysis of NPET 1 post-irradiation (see FIG. 49). The formation of benzyl (5-(hydroxyamino)-5-oxopentyl)carbamate and the nitrosoketone fluorescent by-product was confirmed by ¹H NMR in CD₃CN:1M HCl (4:1) before and after irradiation with a 365 nm UV lamp (30-watt 4-core LED) for 8 minutes. Complete HA release from NPET 1 was indicated by the disappearance of the benzylic proton in the position ortho to the nitro group. Before photolysis, this signal displayed a quartet splitting at 5.41 ppm. After photolysis and the formation of the nitrosoketone by-product, the signal disappeared due to the loss of that hydrogen. Additionally, the hydrogen from the methyl group at 1.53 ppm deshielded and shifted to 2.62 ppm as a singlet. The ¹H-NMR experiment revealed that photo-conversion of NPETs 1 and 2 resulted in a quantitative release of HA (>95%). FT-IR spectra further confirmed the presence of key functional groups (FIGS. 77 and 78).

### Stability Testing of NPET PPGs 1 and 2

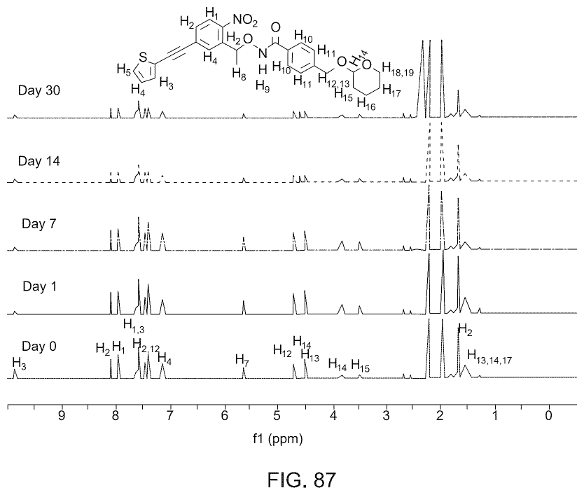

Following the completion of photolysis studies for NPETs 1 and 2, the stability of both PPGs were assessed using time-course ¹H-NMR experiments in CD₃CN to evaluate their shelf life under ambient conditions. NPET 1 showed minimal degradation after one day, with progressive degradation observed over 30 days (FIG. 51). A similar stability study was conducted for NPET 2 in CD₃CN (FIG. 87), which revealed minimal degradation after one day. Notably, even after thirty days, NPET 2 exhibited negligible degradation, with all proton signals in the ¹H-NMR spectra remaining clearly identifiable. These results demonstrate that NPET 2 remain stable under ambient conditions for at least 30 days. Time-course ¹H-NMR data indicates that NPET 2 exhibits greater stability than NPET 1, likely due to the aromatic phenyl ring directly attached to the HA moiety. Aromatic rings are known to absorb UV/visible light without undergoing decomposition and dissipate absorbed energy through non-destructive relaxation pathways, thereby helping prevent photodegradation.

FIG. 87 shows ¹H NMR spectral overlay (in CD₃CN, 400 MHz) of NPET 2, stability test over thirty days under ambient conditions.

### Computational Investigation of the Reactant, Product, and Possible Intermediates of the NPET PPG Cleavage

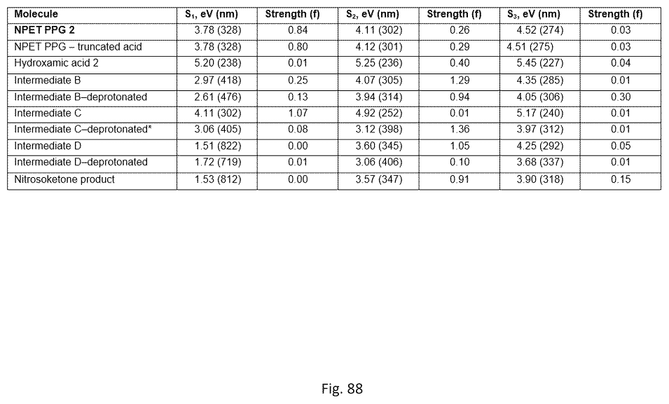

To investigate the origin of the ˜310 nm, ˜345 nm, and ˜400 nm bands in FIG. 83(A) and 63 spectra, TD-DFT calculations were conducted on the putative reactant, product, and some intermediates of the photo-cleavage reaction. For the intermediates, B, C, and D from FIG. 85 and their deprotonated forms were modelled. NPET 2 and associated products were focused on, assuming that the R group of the HA does not strongly influence the absorption wavelengths. Calculations were carried out using the wB97X-D functional and the cc-pVDZ basis set and a continuum solvation model to account for acetonitrile. The choice of functional, as well as tests using a different functional are included in the SI Computational Details section (FIG. 79). All calculations were carried out using Q-Chem 6.0. FIG. 88 presents the vertical excitation energies (VEEs) for some of the low-lying excited states.

First, the UV/visible excitation energy was simulated for the full NPET 2 model. A first excited state at 328 nm was obtained, in reasonably good agreement with the experimental λₘₐₓ (340-345 nm). The model was truncated by replacing the R group of the NPET PPG in FIG. 85 with a methyl group and repeated the calculation. When it was verified that such a truncation has a limited effect on the low-lying excitation energies, the same truncation was applied for modeling intermediates B, C, and D. The excitation energies of the hydroxamic acid were computed to verify that it has no absorption in the near-UV or visible range, as expected for a molecule that contains a single aromatic ring.

The appearance of an intermediate state with an absorption at 395 nm after irradiation warrants further investigation, so the low-lying excited states of intermediates B, C, and D, and their deprotonated forms were computed. Out of all the intermediates, only a few present a potentially red-shifted absorption compared to the nitrosoketone product. Intermediate B, the aci-nitro tautomer, has a first excited state (S₁) predicted computationally at near 418 nm, which would be in reasonable agreement with the experimental wavelength observed experimentally (395-400 nm). Previously reported transient absorption studies also find that such an intermediate has an absorption close to 400 nm. However, in the nitroaromatic compounds studied, the intermediate B was found to be very short-lived. Here, it could be that electronic factors contribute to the stability of intermediate B, allowing it to be longer lived. That said, the aci-nitro group is likely to be deprotonated to the nitronate form in the presence of water, but calculations indicate that this will give rise to a further redshift in the absorption.

TD-DFT calculations on the cyclic intermediate C indicate that it does not absorb near 395 nm, although the S₁ and S₂ states of its deprotonated form might (similar energy absorption wavelengths: ˜405 and 398 nm, respectively). When attempting to optimize the deprotonated form of intermediate C in PCM, the structure spontaneously rearranged to deprotonated D form, which is energetically downhill (a frequency calculation on the two structures indicates that intermediate D is lower by 1.34 kcal/mol than intermediate C). Intermediate D, also a nitroso compound, has similar absorption properties to the nitrosoketone fluorescent product. The computations predict that the protonated form of intermediate D has a similar absorption wavelength as the nitrosoketone. The deprotonated form has a red-shifted and bright S₂ state compared to the nitrosoketone and may explain the experimentally red-shifted band at 395 nm, but this would involve deprotonation of the hemiacetal hydroxy group, which is not expected to be acidic.

There is a possibility that the 310 nm band and shoulder may be explained by intermediate D; while the energies of the S₂ and S₃ states resemble those of the nitrosoketone product, the oscillator strength of the S₂ state is larger while S₃ is smaller.

FIG. 88 shows a table showing Time-Dependent Density Functional Theory (TD-DFT) computed vertical excitation energies (VEEs) for the first three singlet excited states computed with wB97X-D in acetonitrile phase change materials (PCM). Energies are provided in units of eV (and in nm in parentheses). The associated oscillator strength for each excitation is also shown. The truncated model of NPET 2 replaces the R group of the NPET PPG in Scheme 2 with a methyl group. The same truncation was applied for computing intermediates B, C, and D. Hydroxamic acid 2 is the photo-cleavage product for NPET 2.

In FIG. 88, “*” indicates that the deprotonated form of C could be optimized in vacuo but was unstable when optimized in PCM (it rearranged to form deprotonated intermediate D). Therefore, this TD-DFT calculation was carried out using PCM for a structure that was optimized in the gas phase.

This is consistent with intermediate D having absorption bands at the same position of the nitrosoketone absorption spectrum (FIG. 84A), but with different intensities. This explanation would also be consistent with the experimental observation that the hemiacetal is typically the long-lived intermediate in these photoreactions and that D is the last intermediate before photo-cleavage is completed.

The excited states of the nitrosoketone product were analyzed. The TD-DFT calculations using both functionals revealed a very low-lying excited state (S₁). This state has a very low oscillator strength, but if it were bright, its absorption would have appeared at around 812 nm in the UV/visible spectrum. Instead, it was found that the experimentally measured spectral peak at ˜310 nm and shoulder spanning ˜350-450 nm (FIG. 83(A) and 63) are due to absorption to the third and second singlet excited states (S₃ and S₂), respectively. While the calculations are again not in exact quantitative agreement with the experiment, they correctly predict a blue-shifted band and a red-shifted shoulder on either side of the reactant absorption band. The present disclosure also notes that the calculations indicate that S₂ is more intense than S₃, while experimentally the S₃ band at 310 nm appears more intense. However, oscillator strength calculations can often be strongly influenced by the solvent model and nature of the transition. The same TD-DFT calculation carried out in the gas phase, for instance, gives a slightly more intense S₃ band (f=0.50) than S₂ (f=0.40).

Together, the TD-DFT calculations on the intermediates indicate that the short-lived observed experimentally with a λₘₐₓ of ca. 395 nm is not likely to be the acetal C, but may be either intermediate B or D.

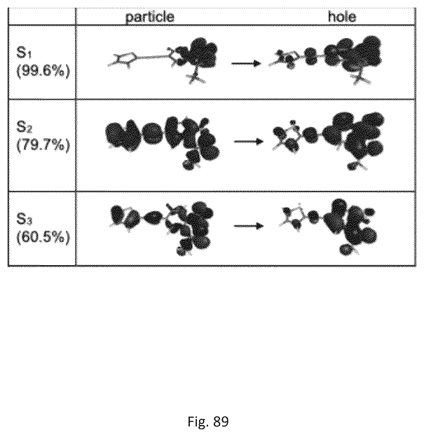

To provide further detail on the electronic nature of these transitions for the nitrosoketone product, Natural Transition Orbitals (NTOs) were computed (FIG. 89). It was found that the S₁ state can best be described as a (n,π*) excitation of a lone pair on the nitroso group. S₂ and S₃, on the other hand are both (π, π*) states involving orbitals delocalized across the thiophene and aromatic nitroso moieties.

FIG. 89 shows representative natural transition orbitals (NTOs) of the first three excited states of the nitrosoketone product. The contribution of each NTO pair to the excitation is indicated as a percentage.

Experimentally, the nitrosoketone fluorescence shows a relatively large Stokes shift with an absorbance at ˜310 nm and emission at ˜475 nm. To better understand the nature of this transition, the geometries were optimized using the gradients of the first and second excited states. The Si state optimization led to a diminishing energy gap and convergence errors consistent with the presence of a non-adiabatic crossing with the ground state. Surprisingly, it was found that the S₂ state is the one responsible for the emission; upon optimization of S₂ and calculation of the vertical emission energy, 2.73 eV was obtained with wB97X-D, corresponding to 454 nm and in good agreement with the observed fluorescence wavelength in FIG. 84B. These results clearly indicate that this fluorescent dye violates Kasha's rule, which instead predicts that fluorescence typically occurs from the lowest excited state (S₁). In the nitrosoketone, the separation between S₁ and S₂ is large enough to prevent fast internal conversion. These calculations agree with previous studies reporting a violation of Kasha's rule in aromatic nitroso compounds.

Photolabile protective groups (PPGs) are particularly desirable because they offer a simple method for real-time monitoring of the cleavage process of main and by-products. To address this need, two new pro-fluorescent ethynylthiophene-based o-NB PPGs, NPETs 1 and 2 were designed, synthesized, and characterized. These PPGs provide an appealing strategy for directly monitoring photolysis through changes in fluorescence intensity, which correlates with the release of photo-cleavage products: hydroxamic acids (HAs) and a pro-fluorescent nitrosoketone by-product. This method addresses the conventional challenges in synthesizing and purifying HA derivatives by facilitating the efficient release of HAS alongside a readily detectable nitrosoketone by-product.

The present disclosure demonstrates the complete release of HA derivatives from NPETs 1 and 2 using UV-Vis spectroscopy, as indicated by a decrease in absorbance intensity accompanied with a blue shift in wavelength. Fluorescence spectroscopy studies further revealed that the cleavage of HA derivatives from NPETs 1 and 2 can be directly monitored and observable to the naked eye. Upon photolysis, NPET 1 exhibits a 4-fold increase in fluorescent intensity, NPET 2 displays a 3-fold increase, both corresponding to the formation of a new emissive nitrosoketone by-product. TD-DFT calculations indicate that this product fluoresces from its second excited singlet state, S₂.

¹H-NMR investigations on NPETs 1 and 2 and their photolysis products confirmed the quantitative release of HA (>95%) and the generation of nitrosoketone by-product. FT-IR analysis of irradiated samples for NPETs 1 and 2 revealed a new —N═O band at 1524 cm⁻¹ with corresponding disappearance of the —NO₂ band, indicating formation of nitroso species, and a new broad absorption band at 3337 cm⁻¹ corresponds to O—H stretch of benzyl (5-(hydroxyamino)-5-oxopentyl)carbamate for cleaved PPG 1, while a broad absorption band at 3467 cm⁻¹ corresponds to the O—H stretch of N-hydroxy-4-(((tetrahydro-2Hpyran-2-yl)oxy)methyl)benzamide for cleaved PPG 2 (see FIGS. 77 and 78). Additionally, ¹H-NMR time-course investigations revealed that NPET 2 exhibits greater stability than NPET 1, showing only minor degradation after 30 days at ambient conditions.

This study highlights the effectiveness of pro-fluorescent, ethynylthiophene-based o-NB PPGs in facilitating precise photoreactions under mild acidic conditions. These findings represent a significant advancement in the development of o-NB PPGs and establish a promising framework for future investigations targeting hard-to-synthesize but spectroscopically well-suited functional groups like carboxylic acids, sulfonates, and phosphates in derivatization targets for the development of absorption-tuned PPGs.

### EXAMPLE CLAUSES

Example Clause 1: A photolabile protecting group compound, comprising: a thiophene ring; a nitrobenzyl group having a nitro group in an ortho position relative to a benzylic carbon; an alkyne linker connecting the thiophene ring to the nitrobenzyl group; and a hydroxamic acid moiety attached to the benzylic carbon through an oxygen atom, wherein upon irradiation with ultraviolet light, the photolabile protecting group compound undergoes photolysis to release hydroxamic acid and generate a fluorescent nitrosoketone byproduct.

Example Clause 2: The photolabile protecting group compound of Example Clause 1, wherein the thiophene ring is connected to the nitrobenzyl group through a 2 -position of the thiophene ring.

Example Clause 3: The photolabile protecting group compound of Example Clause 1 or Example Clause 2, wherein the nitrobenzyl group has a methyl group attached to the benzylic carbon.

Example Clause 4: The photolabile protecting group compound of any one of Example Clauses 1-3, wherein the hydroxamic acid moiety is selected from the group consisting of benzyl (5-(hydroxyamino)-5-oxopentyl)carbamate and N-hydroxy-4-(((tetrahydro-2H-pyran-2-yl)oxy)methyl)benzamide.

Example Clause 5: The photolabile protecting group compound of any one of Example Clauses 1-4, wherein the compound has an absorption maximum between 340 nanometer (nm) and 360 nm.

Example Clause 6: The photolabile protecting group compound of any one of Example Clauses 1-5, wherein upon photolysis, the fluorescent nitrosoketone byproduct has an emission maximum between 440 nm and 500 nm.

Example Clause 7: The photolabile protecting group compound of any one of Example Clauses 1-6, wherein the photolysis results in a 3-fold to 4-fold increase in fluorescence intensity that is observable with a naked eye.

Example Clause 8: A method of synthesizing a hydroxamic acid, comprising: providing a photolabile protecting group compound having a thiophene ring connected to a nitrobenzyl group through an alkyne linker, wherein a hydroxamic acid moiety is attached to a benzylic carbon of the nitrobenzyl group through an oxygen atom; irradiating the photolabile protecting group compound with ultraviolet light at a wavelength of about 365 nm; and releasing the hydroxamic acid from the photolabile protecting group compound while simultaneously generating a fluorescent nitrosoketone byproduct that provides real-time monitoring of the releasing the hydroxamic acid from the photolabile protecting group compound.

Example Clause 9: The method of Example Clause 8, wherein the irradiating the photolabile protecting group compound is performed in a solvent mixture comprising acetonitrile and hydrochloric acid.

Example Clause 10: The method of Example Clause 8 or Example Clause 9, wherein the solvent mixture comprises a 4:1 volume ratio of acetonitrile to 1M hydrochloric acid.

Example Clause 11: The method of any one of Example Clauses 8-10, wherein the thiophene ring is connected to the nitrobenzyl group through a 2-position of the thiophene ring.

Example Clause 12: The method of any one of Example Clauses 8-11, wherein the nitrobenzyl group has a methyl group attached to the benzylic carbon.

Example Clause 13: The method of any one of Example Clauses 8-12, wherein the fluorescent nitrosoketone byproduct exhibits a 3-fold to 4-fold increase in fluorescence intensity compared to the photolabile protecting group compound before the irradiating the photolabile protecting group compound with the ultraviolet light.

Example Clause 14: The method of any one of Example Clauses 8-13, wherein the fluorescent nitrosoketone byproduct has an emission maximum between 440 nm and 500 nm.

Example Clause 15: A compound having a structure: wherein R is selected from the group consisting of alkyl chains, aromatic groups, and protected aromatic groups, and wherein the compound exhibits pro-fluorescent properties upon photolysis with ultraviolet light to release a hydroxamic acid derivative and generate a detectable fluorescent nitrosoketone byproduct.

Example Clause 16: The compound of Example Clause 15, wherein the R is an alkyl chain having 4 to 6 carbon atoms.

Example Clause 17: The compound of Example Clause 15 or Example Clause 16, wherein the R is an aromatic group selected from the group consisting of phenyl groups and substituted phenyl groups.

Example Clause 18: The compound of any one of Example Clauses 15-17, wherein the aromatic group comprises a tetrahydro-2H-pyran-2-yl protecting group.

Example Clause 19: The compound of any one of Example Clauses 15-18, wherein the compound has an absorption maximum between 340 nanometers (nm) and 345 nm and exhibits a molar (M) absorption coefficient of at least 2.0 104 M-1 centimeter(cm) 1.

Example Clause 20: The compound of any one of Example Clauses 15-19, wherein upon photolysis the detectable fluorescent nitrosoketone byproduct exhibits an emission maximum between 440 nm and 500 nm with a quantum yield of at least 0.20%.

The foregoing disclosure provides illustration and description but is not intended to be exhaustive or to limit the implementations to the precise form disclosed. Modifications may be made in light of the above disclosure or may be acquired from practice of the implementations. As used herein, the term “component” is intended to be broadly construed as hardware, firmware, or a combination of hardware and software. It will be apparent that systems and/or methods described herein may be implemented in different forms of hardware, firmware, and/or a combination of hardware and software. The actual specialized control hardware or software code used to implement these systems and/or methods is not limiting of the implementations. Thus, the operation and behavior of the systems and/or methods are described herein without reference to specific software code-it being understood that software and hardware can be used to implement the systems and/or methods based on the description herein. As used herein, satisfying a threshold may, depending on the context, refer to a value being greater than the threshold, greater than or equal to the threshold, less than the threshold, less than or equal to the threshold, equal to the threshold, and/or the like, depending on the context. Although particular combinations of features are recited in the claims and/or disclosed in the specification, these combinations are not intended to limit the disclosure of various implementations. In fact, many of these features may be combined in ways not specifically recited in the claims and/or disclosed in the specification.

Although each dependent claim listed below may directly depend on only one claim, the disclosure of various implementations includes each dependent claim in combination with every other claim in the claim set. No element, act, or instruction used herein should be construed as critical or essential unless explicitly described as such. Also, as used herein, the articles “a” and “an” are intended to include one or more items and may be used interchangeably with “one or more.” Further, as used herein, the article “the” is intended to include one or more items referenced in connection with the article “the” and may be used interchangeably with “the one or more.” Furthermore, as used herein, the term “set” is intended to include one or more items (e.g., related items, unrelated items, a combination of related and unrelated items, and/or the like), and may be used interchangeably with “one or more.” Where only one item is intended, the phrase “only one” or similar language is used. Also, as used herein, the terms “has,” “have,” “having,” or the like are intended to be open-ended terms. Further, the phrase “based on” is intended to mean “based, at least in part, on” unless explicitly stated otherwise. Also, as used herein, the term “or” is intended to be inclusive when used in a series and may be used interchangeably with “and/or,” unless explicitly stated otherwise (e.g., if used in combination with “either” or “only one of”).

What is claimed is:

1. A photolabile protecting group compound, comprising:
a thiophene ring;
a nitrobenzyl group having a nitro group in an ortho position relative to a benzylic carbon;
an alkyne linker connecting the thiophene ring to the nitrobenzyl group; and
a hydroxamic acid moiety attached to the benzylic carbon through an oxygen atom,
wherein upon irradiation with light, the photolabile protecting group compound undergoes photolysis to release hydroxamic acid and generate a fluorescent nitrosoketone byproduct.

2. The photolabile protecting group compound of claim 1, wherein the thiophene ring is connected to the nitrobenzyl group through a 2-position of the thiophene ring.

3. The photolabile protecting group compound of claim 1, wherein the nitrobenzyl group has a methyl group attached to the benzylic carbon.

4. The photolabile protecting group compound of claim 3, wherein the hydroxamic acid moiety is selected from the group consisting of benzyl (5-(hydroxyamino)-5-oxopentyl)carbamate and N-hydroxy-4-(((tetrahydro-2H-pyran-2-yl)oxy)methyl)benzamide.

5. The photolabile protecting group compound of claim 1, wherein the compound has an absorption maximum between 340 nanometer (nm) and 360 nm.

6. The photolabile protecting group compound of claim 5, wherein upon photolysis, the fluorescent nitrosoketone byproduct has an emission maximum between 440 nm and 500 nm.

7. The photolabile protecting group compound of claim 6, wherein the photolysis results in a 3-fold to 4-fold increase in fluorescence intensity that is observable with a naked eye.

8. A method of synthesizing a hydroxamic acid, comprising:
providing a photolabile protecting group compound having a thiophene ring connected to a nitrobenzyl group through an alkyne linker, wherein a hydroxamic acid moiety is attached to a benzylic carbon of the nitrobenzyl group through an oxygen atom;
irradiating the photolabile protecting group compound with light at a wavelength of in the range of 365 nm-400 nm; and
releasing the hydroxamic acid from the photolabile protecting group compound while simultaneously generating a fluorescent nitrosoketone byproduct that provides real-time monitoring of the releasing the hydroxamic acid from the photolabile protecting group compound.

9. The method of claim 8, wherein the irradiating the photolabile protecting group compound is performed in a solvent mixture comprising acetonitrile and hydrochloric acid.

10. The method of claim 9, wherein the solvent mixture comprises a 4:1 volume ratio of acetonitrile to 1M hydrochloric acid.

11. The method of claim 8, wherein the thiophene ring is connected to the nitrobenzyl group through a 2-position of the thiophene ring.

12. The method of claim 11, wherein the nitrobenzyl group has a methyl group attached to the benzylic carbon.

13. The method of claim 8, wherein the fluorescent nitrosoketone byproduct exhibits a 3-fold to 4-fold increase in fluorescence intensity compared to the photolabile protecting group compound before the irradiating the photolabile protecting group compound with the ultraviolet light.

14. The method of claim 13, wherein the fluorescent nitrosoketone byproduct has an emission maximum between 440 nm and 500 nm.

15. A compound having a structure: wherein R is selected from the group consisting of alkyl chains, aromatic groups, and protected aromatic groups, and wherein the compound exhibits pro-fluorescent properties upon photolysis with ultraviolet light to release a hydroxamic acid derivative and generate a detectable fluorescent nitrosoketone byproduct.

16. The compound of claim 15, wherein the R is an alkyl chain having 4 to 6 carbon atoms.

17. The compound of claim 15, wherein the R is an aromatic group selected from the group consisting of phenyl groups and substituted phenyl groups.

18. The compound of claim 17, wherein the aromatic group comprises a tetrahydro-2H-pyran-2-yl protecting group.

19. The compound of claim 15, wherein the compound has an absorption maximum between 340 nm and 345 nm and exhibits a molar (M) absorption coefficient of at least 2.0×10⁴ M⁻¹ centimeter (cm)⁻¹.

20. The compound of claim 19, wherein upon photolysis the detectable fluorescent nitrosoketone byproduct exhibits an emission maximum between nm and 500 nm with a quantum yield of at least 0.20%.
